SEQUENCE LISTING (2 sequences)

SEQ ID NO: 1 - protein, 276 aa, Homo sapiens
DSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 2 - protein, 277 aa, Homo sapiens
ADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

(19) United States
(12) Patent Application Publication
(10) Pub. No.: US 2009/0214506 A1
(43) Pub. Date: Aug. 27, 2009

(54) Use of TFPI to Treat Severe Bacterial Infections

(75) Inventors: Stephen F. Hardy, San Francisco, CA (US); Yumin Dai, Emeryville, CA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Emeryville, CA (US)

(21) Appl. No.: 11/918,662

(22) PCT Filed: May 8, 2006
(86) PCT No.: PCT/US2006/017956

§ 371 Date: Jan. 26, 2009

Related U.S. Application Data
(60) Provisional application No. 60/678,465, filed on May 6, 2005.

(51) Int. Cl. A61K 38/44; A61K 38/16

(52) U.S. Cl. 424/94.4; 514/12

(57) ABSTRACT

Methods for prophylactically or therapeutically treating a patient at risk of developing or diagnosed as having a severe bacterial infection involving administration of tissue factor pathway inhibitor (TFPI) or a TFPI analog to patients suffering from or at risk of developing this condition. The methods involve the use of continuous intravenous infusion of TFPI or a TFPI analog at low doses to avoid adverse side effects.

### BACKGROUND OF THE INVENTION

Severe bacterial infections can lead to a variety of complications, including life-threatening sepsis. There is a continuing need in the art: for effective methods of treating severe bacterial infections and/or reducing the risk of mortality from severe bacterial infections.

### SUMMARY OF THE INVENTION

One embodiment of the present invention is a method of treating a patient at risk of developing or diagnosed as having a severe bacterial infection, comprising administering TFPI or a TFPI analog to a patient in need thereof. In some embodiments, the severe bacterial infection causes pneumonia, bacteremia, deep tissue infection, skin infection, soft tissue infection, periodontal infection, peritonitis, surgical infection, or meningitis. In additional embodiments, the pneumonia is community-acquired pneumonia or hospital acquired pneumonia and can be caused by S. pneumoniae.

Another embodiment is a method of reducing the risk of mortality from a severe bacterial infection, comprising administering a pharmaceutical composition comprising TFPI or a TFPI analog to a patient in need thereof. In some embodiments, the severe bacterial infection causes pneumonia, bacteremia, deep tissue infection, skin infection, soft tissue infection, periodontal infection, peritonitis, surgical infection, or meningitis. In additional embodiments, the pneumonia is community-acquired pneumonia or hospital acquired pneumonia and can be caused by S. pneumoniae.

Other embodiments include any of the above embodiments wherein said patient meets any of the following criteria: a blood IL-6 level below 3,200 pg/ml; an International Normalized Ratio (INR) below 2.5; an acute physiology score (APS) less than 26; an Acute Physiology And Chronic Health Evaluation (APACHE II) score less than 38; and a MODS score greater than 18.

Other embodiments include any of the above embodiments wherein said TFPI or TFPI analog is non-glycosylated. Other embodiments include the above embodiments wherein less than about 12% of the TFPI or TFPI analog molecules are modified species, wherein the modified species include one or more of the following: an oxidized TFPI or TFPI analog molecule, as detected by reverse phase chromatography; a carbamylated TFPI or TFPI analog molecule, as detected by cation exchange chromatography; a deamidated TFPI or TFPI analog molecule, as detected by a Promega ISOQUANT® kit; a TFPI or TFPI analog molecule that comprises a cysteine adduct, as determined by amino acid analysis; aggregated TFPI or TFPI analog molecules, as detected by size exclusion chromatography; and a misfolded TFPI or TFPI analog molecule, as detected by non-denaturing SDS-polyacrylamide gel electrophoresis.

Other embodiments include those above wherein less than about 9% of the TFPI or TFPI analog molecules are oxidized.

Other embodiments include those above wherein less than about 3% of the TFPI or TFPI analog molecules are carbamylated.

Other embodiments include those above wherein less than about 9% of the TFPI or TFPI analog molecules are deamidated.

Other embodiments include those above wherein less than about 2% of the TFPI or TFPI analog molecules comprise a cysteine adduct.

Other embodiments include those above wherein less than about 3% of the TFPI or TFPI analog molecules are aggregated.

Other embodiments include those above wherein less than about 3% of the TFPI or TFPI analog molecules are misfolded.

Other embodiments include those above wherein the TFPI or TFPI analog is prepared from a lyophilized composition comprising TFPI or a TFPI analog. Other embodiments include those above wherein the TFPI or TFPI analog is administered as a formulation comprising arginine. Other embodiments include those above wherein the TFPI or TFPI analog may be administered as a formulation comprising citrate.

Other embodiments include those above wherein the pharmaceutical composition comprises 0.01 to 1.0 mg/ml TFPI or TFPI analog. Other embodiments include those above wherein the pharmaceutical composition comprises 150-450 mM L-arginine. Other embodiments include those above wherein the pharmaceutical composition comprises 0.1-50 mM L-methionine. Other embodiments include those above wherein the pharmaceutical composition comprises 5-50 mM sodium citrate buffer. Other embodiments include those above wherein the pharmaceutical composition has a pH of 5.0-6.0.

Other embodiments include those above wherein the method comprises use of the pharmaceutical composition comprising 0.15±15% mg/ml TFPI or TFPI analog, 300±15% mM L-arginine, 5±15% mM L-methionine, and 20±15% mM sodium citrate buffer at pH 5.5±15%.

Other embodiments include those above wherein the method comprises use of the pharmaceutical composition comprising 0.15±10% mg/ml TFPI or TFPI analog, 300±10% mM L-arginine, 5±10% mM L-methionine, and 20±10% mM sodium citrate buffer at pH 5.5±10%.

Other embodiments include those above wherein the method comprises use of the pharmaceutical composition comprising 0.15±5% mg/ml TFPI or TFPI analog, 300±5% mM L-arginine, 5±5% mM L-methionine, and 20±5% mM sodium citrate buffer at pH 5.5±5%.

Other embodiments include those above wherein the method comprises use of the pharmaceutical composition comprising 0.45±15%-mg/ml TFPI or TFPI analog, 300±15% mM L-arginine, 5±15% mM L-methionine, and 20±15% mM sodium citrate buffer at pH 5.5±15%.

Other embodiments include those above wherein the method comprises use of the pharmaceutical composition comprising 0.45±10% mg/ml TFPI or TFPI analog, 300±10% mM L-arginine, 5±10% mM L-methionine, and 20±10% mM sodium citrate buffer at pH 5.5±10%.

Other embodiments include those above wherein the method comprises use of the pharmaceutical composition comprising 0.45±5% mg/ml TFPI or TFPI analog, 300±5% mM L-arginine, 5±5% mM L-methionine, and 20±5% mM sodium citrate buffer at pH 5.5±5%.

Other embodiments include those above wherein the TFPI or TFPI analog is administered by continuous intravenous infusion at a dose rate equivalent to administration of reference ala-TFPI at a dose rate of less than about 0.66 mg/kg/hr.

Other embodiments include those above wherein the dose rate is equivalent to administration of reference ala-TFPI at a dose rate from about 0.00025 to about 0.1 mg/kg/hr and wherein the TFPI or TFPI analog is administered for at least about 72 hours. Other embodiments include those above wherein the dose rate is equivalent to administration of reference ala-TFPI at a dose rate from about 0.010 to about 0.1 mg/kg/hr. Other embodiments include those above wherein the TFPI or the TFPI analog is administered for at least about 96 hours.

Other embodiments include those above wherein the TFPI or TFPI analog is administered by continuous intravenous infusion to provide a total dose equivalent to administration of reference ala-TFPI at a total dose from about 0.024 to about 4.8 mg/kg. Other embodiments include those above wherein the TFPI or TFPI analog is administered by continuous intravenous infusion at a dose rate equivalent to administration of reference ala-TFPI at a dose rate between about 0.02 to about 1 mg/kg/hr. Other embodiments include those above wherein the TFPI or TFPI analog is administered by continuous intravenous infusion to provide a daily dose equivalent to administration of reference ala-TFPI at a daily dose from about 0.006 mg/kg to about 1.2 mg/kg.

Other embodiments include those above wherein the TFPI or TFPI analog is administered by infusion for a period of 10-200 hours, 10-150 hours, or 24-96 hours.

Other embodiments include those above wherein the patient has not received heparin treatment for at least 8 hours before administration of the TFPI or TFPI analog.

Other embodiments include those above further comprising treating the patient with activated protein C.

Other embodiments include those above wherein the TFPI analog is ala-TFPI.

### DETAILED DESCRIPTION

TFPI is a powerful anticoagulant thought to have anti-inflammatory activity. See EP 0 643 585. TFPI can be used to inhibit angiogenesis associated with, for example, tumors. See EP 0 914 830. We have found that TFPI also has antibacterial activity through its control of inflammatory cytokines which control the innate immune system. We also found that TFPI appears to be degraded as a result of severe bacterial infection, and depletion of TFPI during an ongoing infection compromises this system. Thus, while TFPI plays a vital role in combating infection, endogenous TFPI is altered during a severe infection, which depletes TFPI's therapeutic function.

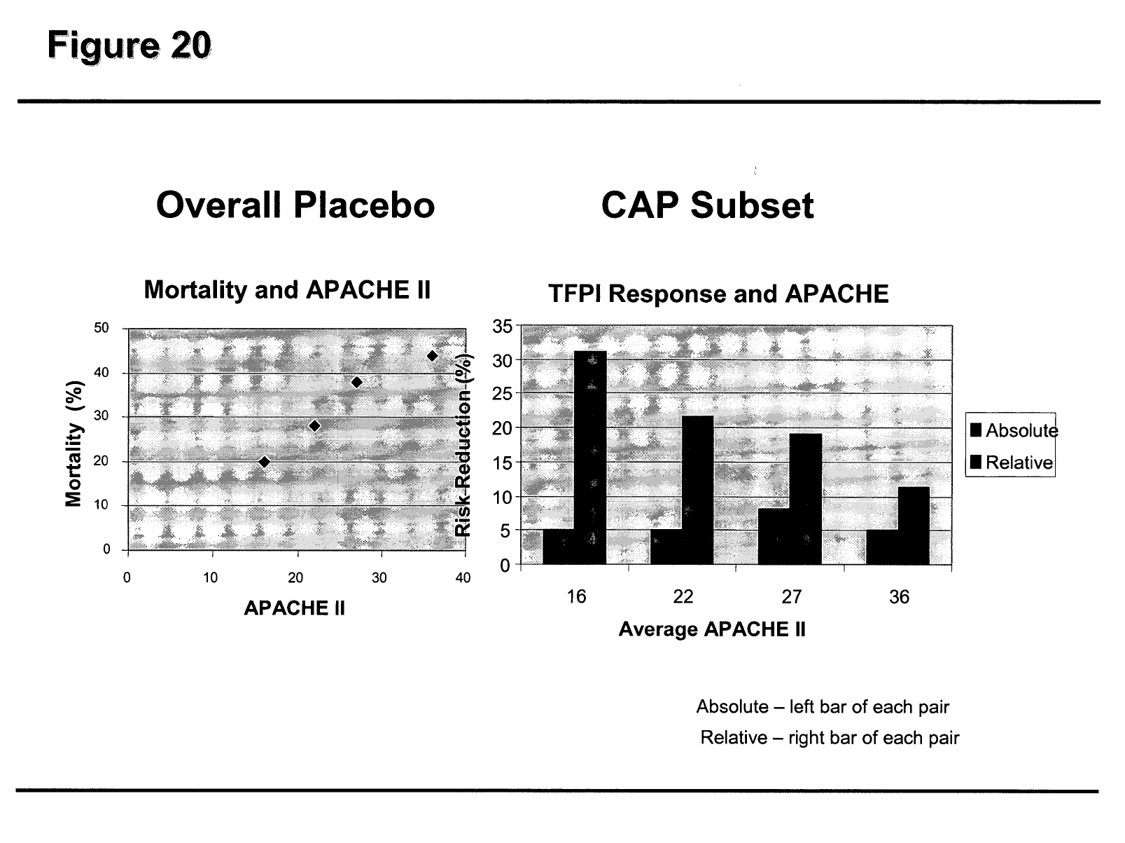

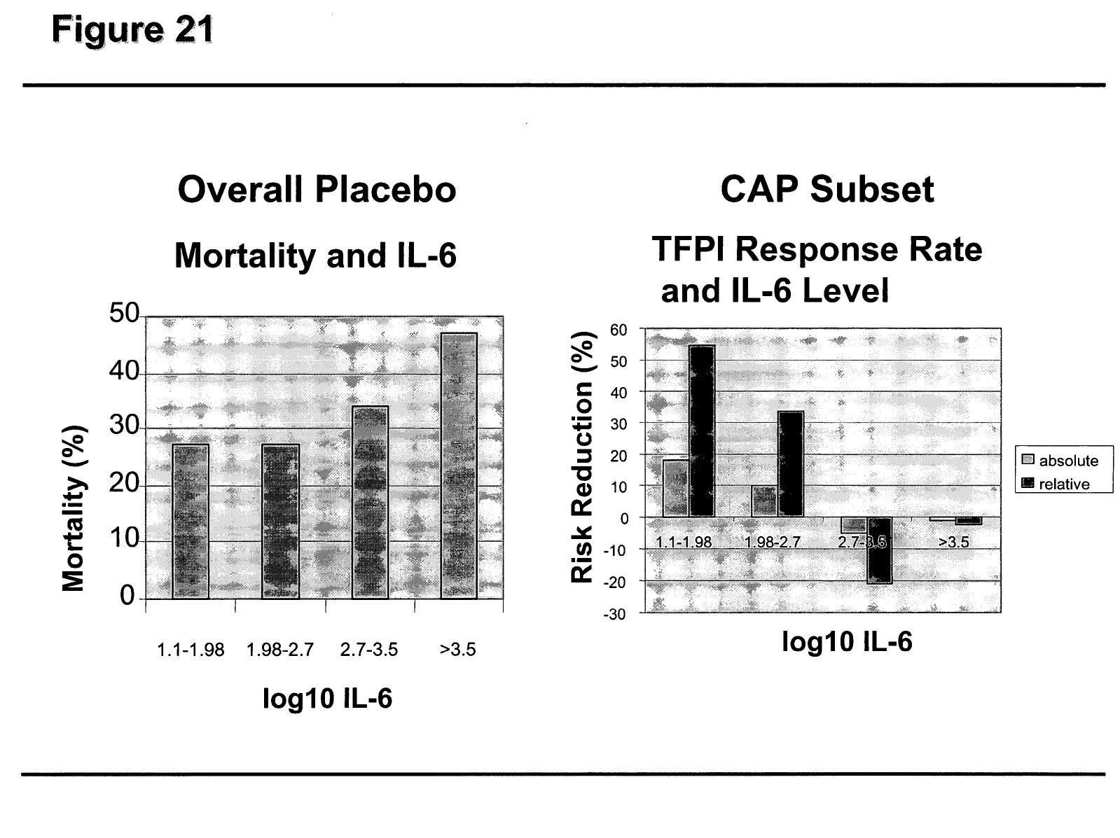

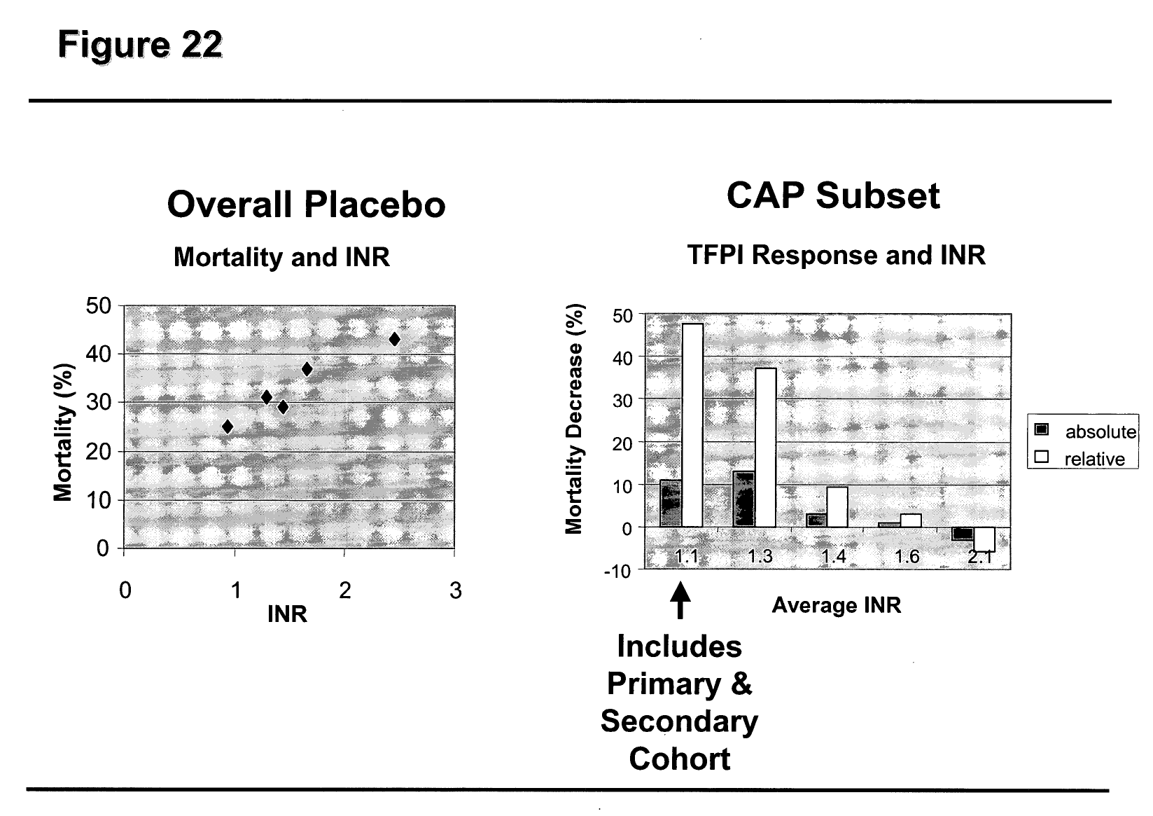

Several factors predict mortality in groups of septic patients. These include the Acute Physiology And Chronic Health Evaluation (APACHE II) scoring system (Knaus et al., Crit Care Med. 1985;13:818-29), acute physiology score (APS), International Normalized Ratio (INR) (R. S. Riley et al., J. Clin. Lab. Anal. 14:101-114, 2000), and plasma IL-6 concentration. In a population of patients with community acquired pneumonia (CAP), we had previously observed that TFPI was effective in reducing mortality. The relative risk reduction is determined as (100× the absolute risk reduction)/placebo mortality rate. When we divided the CAP patients by severity using APACHE II scores (FIG. 20), acute physiology scores, INR, (FIG. 22) or plasma IL-6 levels, (FIG. 21) we found that benefit from TFPI treatment included patients with the lowest placebo mortality rates. For example, in the CAP subset divided by APACHE II scores, there was a 5% mortality risk reduction at APACHE II score of 16, which means a 32% relative risk reduction. FIG. 20 demonstrate reduced mortality risks in patents with average APACHE II scores of 36 or below. Baseline TFPI serum concentrations predicted both mortality and efficacy.

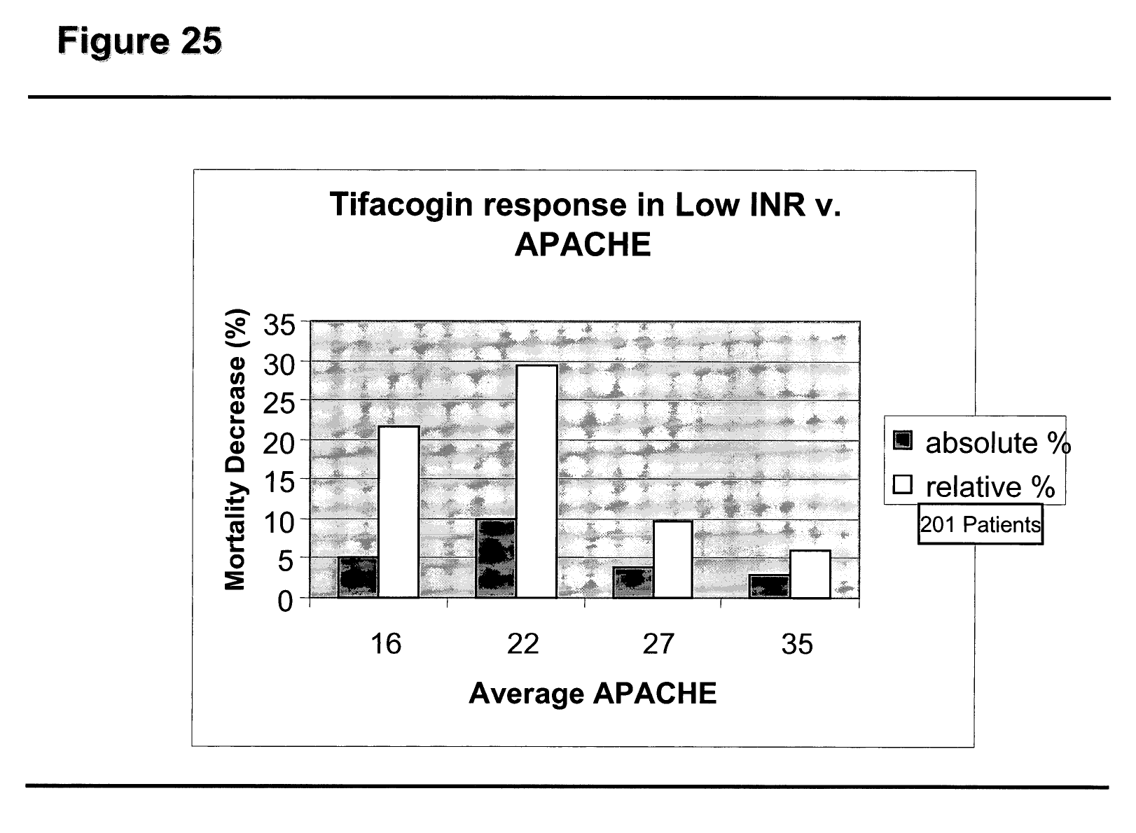

Patients in a substudy with INR levels below 1.2 had a lower risk of mortality than those in the high INR primary study (18% vs. 34% overall) and ala-TFPI was effective in the substudy with INR levels below 1.2 (FIG. 25). In the CAP subset of patients with INR levels below an average of 2.1 benefited from ala-TFPI (FIG. 21). This is also true for patients treated with ala-TFPI without heparin treatment.

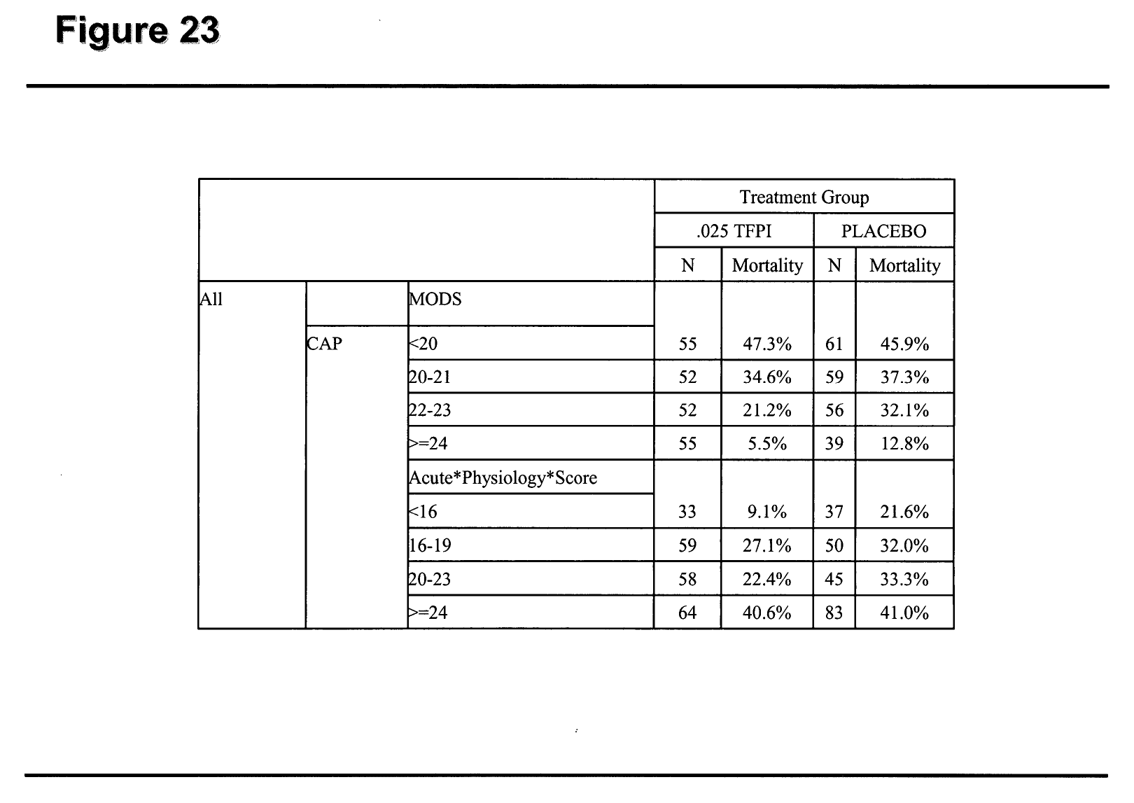

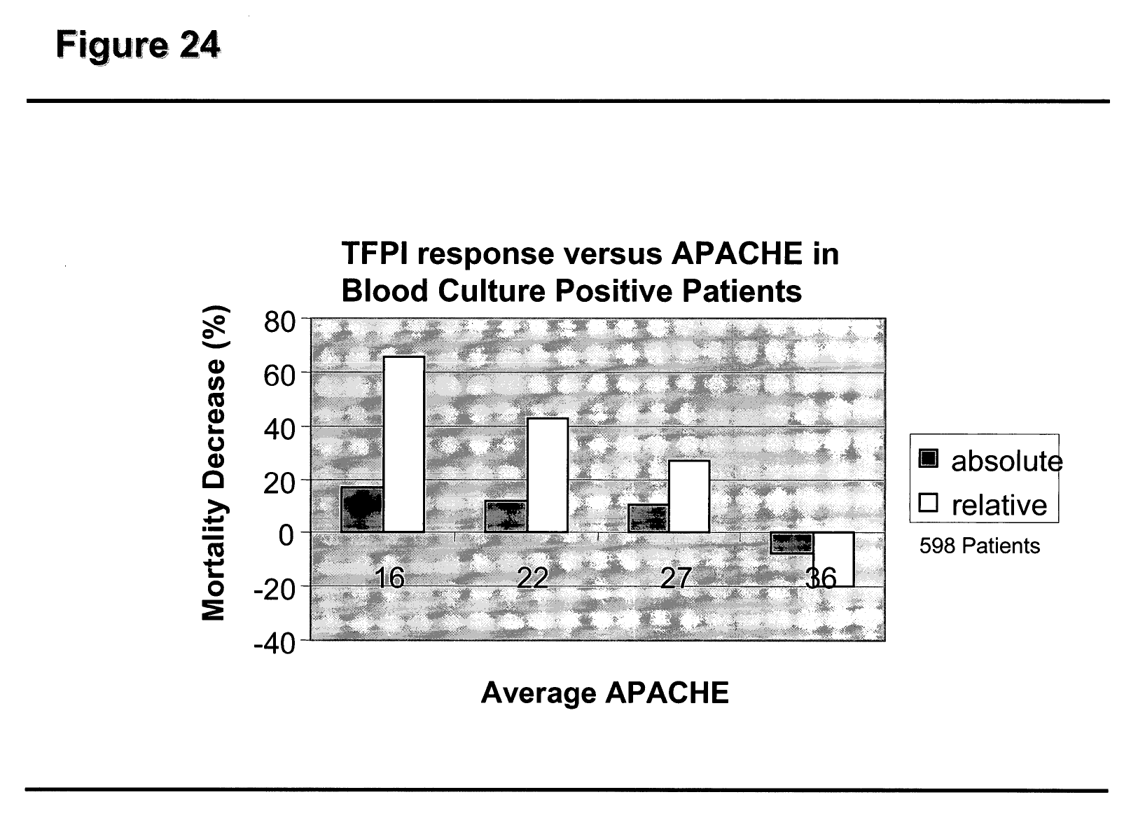

FIG. 20 shows the expected correlations between APACHE II and mortality in the overall and community acquired pneumonia (CAP) placebo groups (FIG. 20). When the CAP cohort into APACHE II quartiles, we observed that there was efficacy in all APACHE II quartiles. Similarly, APS, INR and IL-6 were all positively correlated with mortality. In each case ala-TFPI improved survival in the lowest risk groups as measured by these parameters (FIGS. 21, 22, 23). TFPI has efficacy in patients who had positive bacterial cultures from their blood (FIG. 24). Again TFPI was broadly active in the first 3 quartiles when the patients were segregated by APACHE scores.

Thus, treatment with exogenous TFPI has therapeutic benefit for patients who are at risk of developing or diagnosed as having a severe bacterial infection and is useful for lowering the risk of mortality from such infections. Severe bacterial infections are those infections generally requiring acute medical care, especially hospitalization. Severe bacterial infections generally need antiinfective treatment and supportive medical care or active medical intervention. Common severe bacterial infections include, but are not limited to, pneumonia (including hospital acquired pneumonia and community acquired pneumonia), bacteremia, deep tissue infection, skin infection, soft tissue infection, periodontal infection, peritonitis, surgical infection, and meningitis.

Patients who can be treated according to the invention meet one or more of the following criteria: (a) a blood IL-6 level below 3,200 pg/ml (e.g., blood IL-6 levels below 3,000, 2,500, 2,000, 1,500, 1,000, 500, 250, 100 or 10); (b) an International Normalized Ratio (INR) below 2.5 (e.g., an INR below 2.4, 2.3, 2.2, 2.1, 1.9, 1.8, 1.7, 1.6, 1.5, 1.4, 1.3, 1.2, or 1.1); (c) an acute physiology score (APS) less than 26 (e.g., an APS score less than 25, 24, 23, 22, 21, 20, 19, 18, 17, or 16, or between 16-19, or between 20-23); (d) an Acute Physiology And Chronic Health Evaluation (APACHE II) score less than 38 (e.g., APACHE II score less than 37, 36, 35, 34, 33, 32, 31, 30, 29, 28, 27, 26, 25, 24, 23, 22, 21, 20, 19, 18, 17, or 16); and (e) a Multiple Organ Dysfunction (MODS) score greater than 18 (e.g., a MODS score greater than 19, 20, 21, 22, 23, or 24, or between than 20-21, or between 22-23).

In one embodiment, the patient has a blood IL-6 level below 3,200 pg/ml. In another embodiment, the patient has an International Normalized Ratio (INR) below 2.5. In yet another embodiment, the patient has an acute physiology score (APS) less than 26. In still another embodiment, the patient has an Acute Physiology And Chronic Health Evaluation (APACHE II) score less than 38. In yet another embodiment, the patient has a MODS score greater than 18.

### TFPI and TFPI Analogs

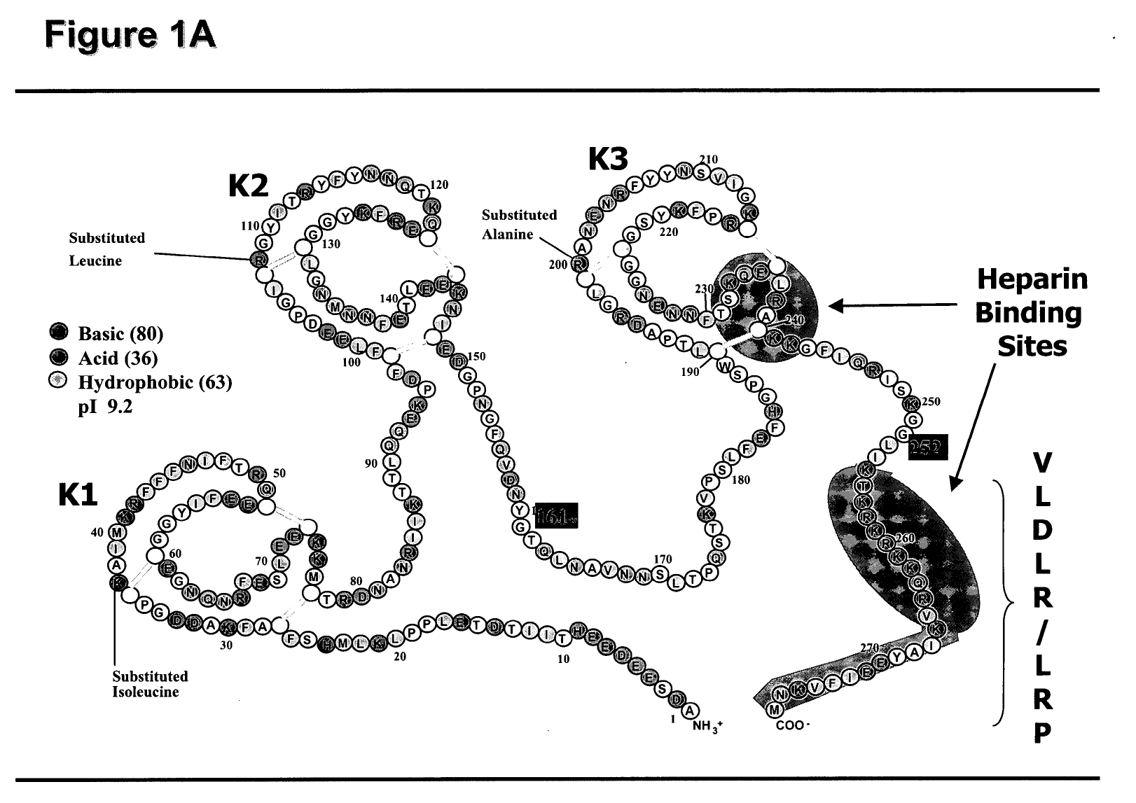

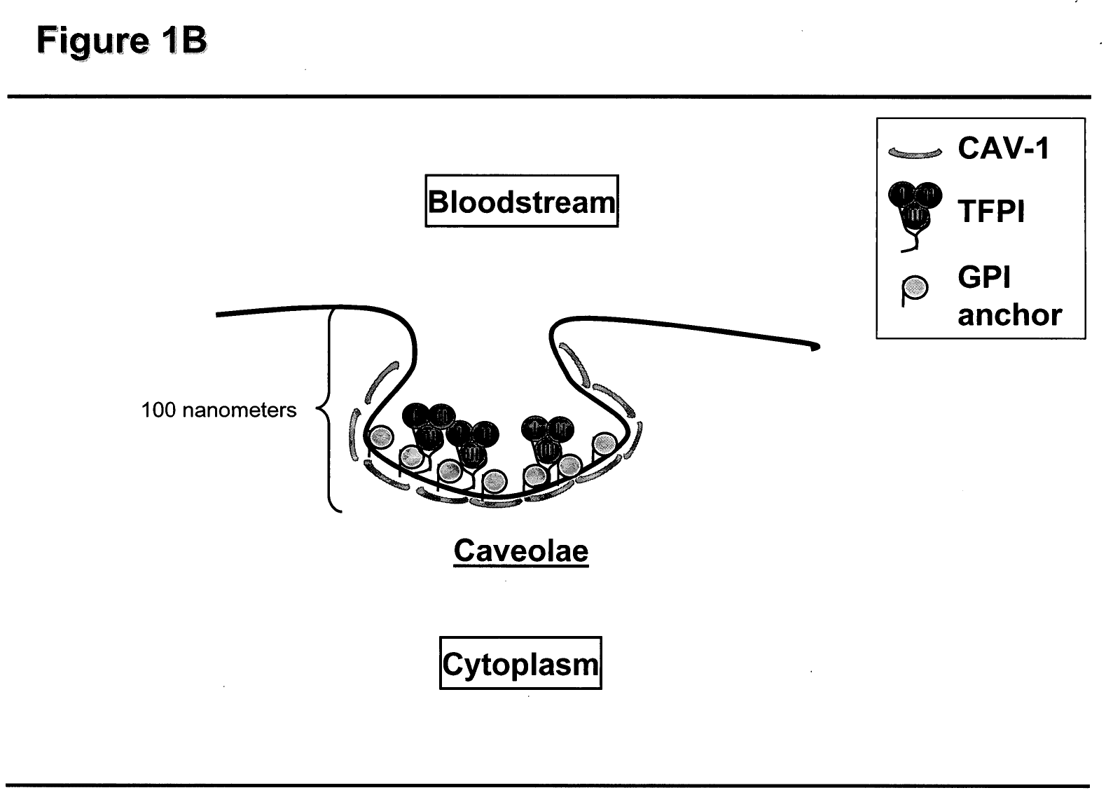

TFPI was initially isolated as a naturally occurring anticoagulant. The protein has several principal domains (FIG. 1): three serine protease inhibitor domains of the Kunitz type (K1, K2 and K3), an N-terminal domain, and a C-terminal domain (CTD). The K1 domain inhibits clotting factor VIIa-tissue factor (TF) complex. The K2 domain inhibits factor Xa. Thus far no serine protease has been associated with K3, however recent experiments suggest that K3 functions in binding TFPI to a GPI anchored receptor on cell surfaces. Piro & Broze, Circulation. 2004 Dec. 7;110(23):3567-72. The CTD is also involved in cell association, heparin binding, and optimal Xa inhibition.

TFPI is naturally produced in multiple cell types in blood and in other tissues. It is believed that most TFPI is made in endothelial cells lining the blood vessels. A fraction of TFPI is found in blood. Most of this plasma TFPI is covalently modified by disulfide exchange with other plasma proteins or cleavage removing the CTD. These forms of TFPI have less activity in biochemical assays and do not bind cells efficiently suggesting that that plasma TFPI is inactive. In agreement with this studies have shown that the half life of plasma TFPI is very short and that it is efficiently removed by the liver.

The various cell associated forms of TFPI are the full length monomeric protein associated with maximum activity iii vitro. On endothelial cells there is a small fraction of TFPI which can be removed by treatment with heparin. Another pool appears to sit in vesicles below the apical surface and is released when the cells are stimulated with thrombin. The majority of cell surface TFPI is found in caveolae, an organelle associated with signaling in other systems. Lupu et al., JBC Papers in Press, published Apr. 6, 2005 as Manuscript M503333200. The TFPI in caveolae appears to be the TFPI that engages TF on endothelial cells. When an endothelial cell is exposed to bacteria it induces TF generally over its surface. The TF binds to VIIa generating Xa. TF:VIIa rapidly relocates to caveolae where it binds to TFPI. In normal endothelial cells there is an excess of TFPI, ensuring that TF-expressing endothelial cells do not induce pathological levels of clotting enzymes. Curiously, once TF translocates to caveolae it becomes stable, suggesting that it has a role to play after it has been neutralized by TFPI.

“TFPI” as used herein refers to the mature serum glycoprotein having the 276 amino acid residue sequence shown in SEQ ID NO:1 and a molecular weight of about 38,000 Daltons. See U.S. Pat. No. 5,106,833. The cloning of the TFPI cDNA is described in Wun et al., U.S. Pat. No. 4,966,852. TFPI used in the invention may be non-glycosylated or glycosylated.

A “TFPI analog” is a derivative of TFPI modified with one or more amino acid additions or substitutions (generally conservative in nature and preferably in non-Kunitz domains or in the C terminal portion of the protein), one or more amino acid deletions (e.g., TFPI fragments), or the addition of one or more chemical moieties to one or more amino acids, so long as the modifications do not destroy TFPI biological activity. Preferably, TFPI analogs comprise all three Kunitz domains. TFPI and TFPI analogs can be either glycosylated or non-glycosylated.

A preferred TFPI analog is N-L-alanyl-TFPI (ala-TFPI), whose amino acid sequence is shown in SEQ ID NO:2. Ala-TFPI is also known under the international drug name “tifacogin.” The amino terminal alanine residue of ala-TFPI was engineered into the TFPI sequence to improve E. coli expression and to effect cleavage of what would otherwise be an amino terminal methionine residue. See U.S. Pat. No. 5,212,091. Other analogs of TFPI are described in U.S. Pat. No. 5,106,833. TFPI analogs possess some measure of the activity of TFPI as determined by a bioactivity assay as described below. A preferred bioactivity assay for TFPI and analogs is the prothrombin time (PT) assay (see below).

TFPI analogs can have amino acid substitutions which are conservative in nature, i.e., substitutions which take place within a family of amino acids which are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 1-15 conservative or non-conservative amino acid substitutions (e.g., 1, 2, 3, 4, 5, 6, 7, 8, 9, 10, 11, 12, 13, 14, or 15), as long as the desired function of the molecule remains intact. Preferred forms of TFPI include TFPI analogs where the Kunitz 1, Kunitz 2 or Kunitz 3 domains correspond exactly to native human TFPI Kunitz domains, and more preferably, where each Kunitz domain correspond exactly to each of the native human TFPI Kunitz domain.

Preferably, TFPI analogs have amino acid sequences which are at least or 95% or more identical to TFPI as shown in SEQ ID NO:1. More preferably, the molecules are 96%, 97%, 98% or 99% identical.

The biological activity of TFPI and TFPI analogs can be determined by the prothrombin assay. Suitable prothrombin assays are described in U.S. Pat. No. 5,888,968 and in WO 96/40784. Briefly, prothrombin time can be determined using a coagulometer (e.g., Coag-A-Mate MTX II from Organon Teknika). A suitable assay buffer is 100 mM NaCl, 50 mM Tris adjusted to pH 7.5, containing 1 mg/ml bovine serum albumin. Additional reagents required are normal human plasma (e.g., “Verify 1” by Organon Teknika), thromboplastin reagent (e.g., “Simplastin Excel” by Organon Teknika), and TFPI standard solution (e.g., 20 μg of 100% pure ala-TFPI (or equivalent thereof) per ml of assay buffer). A standard curve is obtained by analyzing the coagulation time of a series of dilutions of the TFPI standard solution, e.g., to final concentrations ranging from 1 to 5 μg/ml.

For the determination of clotting time, the sample or TFPI standard is first diluted into the assay buffer. Then normal human plasma is added. The clotting reaction is started by the addition of thromboplastin reagent. The instrument then records the clotting time. A linear TFPI standard curve is obtained from a plot of log clotting time vs. log TFPI concentration. The standard curve is adjusted based on the purity of the TFPI standard to correspond to the equivalent TFPI concentration of a 100% pure standard. For example, if the standard is a preparation of ala-TFPI that is 97% biochemically pure (i.e., it contains 3% by weight of molecular species without biological activity of TFPI), then the concentration of each dilution of the standard is multiplied by 0.97 to give the actual concentration of TOPIC Thus, a TFPI standard that is 1.0 μg/ml based on the actual weight per ml of a preparation that is 97% pure will be equivalent to, and treated as, a concentration of 1.0×0.97, or 0.97 μg/ml.

### Obtaining TFPI and TFPI Analogs

TFPI and analogs of TFPI used in the methods of the invention can be isolated and purified from cells or tissues, chemically synthesized, or produced recombinantly in either prokaryotic or eukaryotic cells.

TFPI can be isolated by several methods. For example, cells that secrete TFPI include aged endothelial cells, young endothelial cells that have been treated with TNF for about 3 to 4 days, hepatocytes, and hepatoma cells. TFPI can be purified by conventional methods, including the chromatographic methods of Pedersen et al., 1990, J. Biol. Chem.. 265, 16786-93, Novotny et al., 1989, J. Biol. Chem. 264, 18832-37, Novotny et al., 1991, Blood 78, 394-400, Wun et al., 1990, J. Biol. Chem. 265, 16096-101, and Broze et al., 1987, Proc. Natl. Acad. Sci. USA 84, 1886-90. TFPI appears in the bloodstream and can be purified from blood, see Pedersen et al., 1990.

A TFPI or TFPI variant can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of TFPI or TFPI variants can be separately synthesized and combined using chemical methods to produce a full-length molecule.

TFPI and TFPI analogs may be produced recombinantly as shown in U.S. Pat. No. 4,966,852. For example, the cDNA for the desired protein can be incorporated into a plasmid for expression in prokaryotes or eukaryotes. U.S. Pat. No. 4,847,201 provides details for transforming microorganisms with specific DNA sequences and expressing them. There are many other references known to those of ordinary skill in the art that provide details on expression of proteins using microorganisms. Many of those are cited in U.S. Pat. No. 4,847,201, such as Maniatas et al., 1982, Molecular Cloning, Cold Spring Harbor Press.

A variety of techniques are available for transforming microorganisms and using them to express TFPI and TFPI analogs. The following are merely examples of possible approaches. TFPI DNA sequences must be isolated and connected to the appropriate control sequences. TFPI DNA sequences are shown in U.S. Pat. No. 4,966,852 and can be incorporated into a plasmid, such as pUNC13 or pBR3822, which are commercially available from companies such as Boehringer-Mannheim. Once the TFPI DNA is inserted into a vector, it can be cloned into a suitable host. The DNA can be amplified by techniques such as those shown in U.S. Pat. No. 4,683,202 to Mullis and U.S. Pat. No. 4,683,195 to Mullis et al. TFPI cDNA may be obtained by inducing cells, such as hepatoma cells (such as HepG2 and SKHep) to make TFPI mRNA, then identifying and isolating the MRNA and reverse transcribing it to obtain cDNA for TFPI. After the expression vector is transformed into a host such as E. coli, the bacteria may be fermented and the protein expressed. Bacteria are preferred prokaryotic microorganisms and E. coli is especially preferred. A preferred microorganism useful in the present invention is E. coli K-12, strain MM294 deposited with the ATCC on Feb. 14, 1984 (Accession No. 39607), under the provisions of the Budapest Treaty.

It is also, of course, possible to express genes encoding polypeptides in eukaryotic host cell cultures derived from multicellular organisms. See, for example, Tissue Culture, 1973, Cruz and Patterson, eds., Academic Press. Useful mammalian cell lines include murine myelomas N51, VERO, HeLa cells, Chinese hamster ovary (CHO) cells, COS, C127, Hep G2, and SK Hep. TFPI and TFPI analogs can also be expressed in baculovirus-infected insect cells (see also U.S. Pat. No. 4,847,201, referred to above). See also Pedersen et al., 1990, J. of Biological Chemistry, 265:16786-16793. Expression vectors for eukaryotic cells ordinarily include promoters and control sequences compatible with mammalian cells such as, for example, the commonly used early and later promoters from Simian Virus 40 (SV40) (Fiers, et al., 1978, Nature, 273:113), or other viral promoters such as those derived from polyoma, Adenovirus 2, bovine papilloma virus, or avian sarcoma viruses, or immunoglobulin promoters and heat shock promoters.

General aspects of mammalian cell host system transformations have been described by Axel, U.S. Pat. No. 4,399,216. It now appears also that “enhancer” regions are important in optimizing expression; these are, generally, sequences found upstream of the promoter region. Origins of replication may be obtained, if needed, from viral sources. However, integration into the chromosome is a common mechanism for DNA replication in eukaryotes. Plant cells are also now available as hosts, and control sequences compatible with plant cells such as the nopaline synthase promoter and polyadenylation signal sequences (Depicker, A., et al., 1982, J. Mol. Appl. Gen., 1:561) are available. Methods and vectors for transformation of plant cells are disclosed in WO 85/04899.

Methods which can be used for purification of TFPI and TFPI analogs expressed in mammalian cells include sequential application of heparin-Sepharose, MonoQ, MonoS, and reverse phase HPLC chromatography. See Pedersen et al., supra; Novotny et al., 1989, J. Biol. Chem. 264:18832-18837; Novotny et al., 1991, Blood, 78:394-400; Wun et al., 1990, J. Biol. Chem. 265:16096-16101; Broze et al., 1987, PNAS (USA), 84:1886-1890; U.S. Pat. No. 5,106,833; and U.S. Pat. No. 5,466,783. These references describe various methods for purifying mammalian produced TFPI.

A preferred method of preparing TFPI or TFPI analog molecules is disclosed in WO 05/019265. This method produces preparations of TFPI or TFPI analog molecules in which less than about 12, 11, 10, 9, 8, 7, 6, 5, 4, 3, 2, 1, or 0.5% of the preparation consists of “modified species.” “Modified species” include one or more of the following: an oxidized TFPI or TFPI analog molecule, as detected by reverse phase chromatography; a carbamylated TFPI or TFPI analog molecule, as detected by cation exchange chromatography; a deamidated TFPI or TFPI analog molecule, as detected by a Promega ISOQUANT® kit; a TFPI or TFPI analog molecule that comprises a cysteine adduct, as determined by amino acid analysis; aggregated TFPI or TFPI analog molecules, as detected by size exclusion chromatography; and a misfolded TFPI or TFPI analog molecule, as detected by non-denaturing SDS-polyacrylamide gel electrophoresis. Using the method disclosed in WO 05/019265, preparations of TFPI or TFPI analog molecules can be produced in which less than about 9% of the TFPI or TFPI analog molecules are oxidized, less than about 3% of the TFPI or TFPI analog molecules are carbamylated, less than about 9% of the TFPI or TFPI analog molecules are deamidated, less than about 2% of the TFPI or TFPI analog molecules comprise a cysteine adduct, less than about 3% of the TFPI or TFPI analog molecules are aggregated, and less than about 3% of the TFPI or TFPI analog molecules are misfolded.

TFPI also can be expressed as a recombinant glycosylated protein using mammalian cell hosts, such as mouse C127 cells (Day et al., Blood 76, 153845, 1990), baby hamster kidney cells (Pedersen et al., 1990), Chinese hamster ovary cells, and human SK hepatoma cells. C127 TFPI has been used in animal studies and shown to be effective in the inhibition of tissue factor-induced intravascular coagulation in rabbits (Day et al., supra), in the prevention of arterial reocclusion after thrombolysis in dogs (Haskel et al., Circulation 84:821-827 (1991)), and in reduction of mortality in an E. coli sepsis model in baboons (Creasey et al., J. Clin. Invest. 91:2850 (1993)). Ala-TFPI can be expressed as a recombinant non-glycosylated protein using E. coli host cells. Methods have been described which yield a highly active ala-TFPI by in vitro refolding of the recombinant protein produced in E. coli. See, e.g., WO 96/40784.

TFPI and TFPI analogs also can be produced in bacteria or yeast and subsequently purified. Generally, the procedures shown in U.S. Pat. Nos. 5,212,091; 6,063,764; and 6,103,500 or WO 96/40784 can be employed. Ala-TFPI and other TFPI analogs can be purified, solubilized, and refolded according WO 96/40784 and Gustafson et al., Prot. Express. Pur. 5:233 (1994), which are incorporated herein by reference. For example, when prepared according Example 9 of WO 96/40784, preparations of ala-TFPI may be obtained that contain from about 85% to 90% of the total protein by weight as mature, properly-folded, biologically active ala-TFPI, about 10% to 15% of which has one or more oxidized methionine residues. These oxidized forms have biological activity that is equivalent to the biological activity of underivatized ala-TFPI, as determined by prothrombin assay, and are expected to be active in the invention disclosed herein. The remaining material comprises various modified forms of ala-TFPI, including dimerized, aggregated, and acetylated forms.

TFPI and TFPI analogs can have a significant number of cysteine residues, and the procedure shown in U.S. Pat. No. 4,929,700 is relevant to TFPI refolding. TFPI and analogs can be purified from the buffer solution by various chromatographic methods, such as those mentioned above. If desired, the methods shown in U.S. Pat. No. 4,929,700 may be employed. Any method may be employed to purify TFPI and TFPI analogs that results in a degree of purity and a level of activity suitable for administration to humans.

### Therapeutic Methods and Compositions

TFPI and TFPI analogs are useful to treat patients at risk of developing or diagnosed as having a severe bacterial infection or to lower the risk of mortality from severe bacterial infection for one or a group of patients.

Severe bacterial infections include, for example, “severe pneumonia” as defined according to the guidelines set forth by the. American Thoracic Society. Specifically, severe pneumonia requires a diagnosis of pneumonia and the existence of either a) one of two major criteria, b) two of three minor criteria, or c) two of the four criteria from the British Thoracic Society (Thorax 2001; 56 [suppl IV]:1-64). The major criteria are 1) need for mechanical ventilation and 2) septic shock or need for pressors for >4 hours. The minor criteria are 1) systolic blood pressure ≦90 mmHg, 2) multi-lobar pneumonia, and 3) hypoxemia criterion (PaO₂/FiO₂)<250. The criteria from the British Thoracic Society are 1) respiratory rate ≧30 breaths/minute, 2) diastolic blood pressure ≦60 mmHg, 3) blood urea nitrogen (BUN)>7.0 mM (>19.6 mg/dL) and 4) confusion. As is understood in the art, the hypoxemia criterion (PaO₂/FiO₂) refers to the partial pressure of arterial oxygen to the fraction of inspired oxygen and indicates the level of impairment of gas exchange.

Many patients with severe pneumonia have an infection demonstrable by any means known in the art. These methods include, but are not limited to, detection of a pathogenic organism in a culture of blood or other normally sterile body fluid or tissue by, for example, GRAM stain, culture, histochemical staining, immunochemical assay, or nucleic acid assays. A demonstrable infection also can be evidenced by a chest radiograph consistent with a diagnosis of pneumonia that constitutes the reason for systemic anti-infective therapy, as well as any clinical symptom of infection, including, but not limited to, an increase in respiratory rate >/=30/min or PaCO₂/FiO₂<250, a decrease in blood pressure, and an increase in body temperature.

Formulations of TFPI and TFPI Analogs

Formulations of TFPI and TFPI analogs preferably are administered by intravenous infusions. Essentially continuous intravenous infusion is preferred. Methods to accomplish this administration are known to those of ordinary skill in the art. Infusion can be performed via a central line or a peripheral line. While large fluctuations in the dose rate are to be avoided, short-term deviations from the dose rates of the invention are acceptable provided the resulting plasma level of administered TFPI is within 20% of that expected from a continuous infusion at a constant dose rate according to the preferred embodiments of invention.

Before administration to patients, formulants may be added to TFPI and TFPI analogs. A liquid formulation is preferred. TFPI and TFPI analogs may be formulated at different concentrations, using different formulants, and at any physiologically suitable pH compatible with the route of administration, solubility, and stability of the TFPI protein. A preferred formulation for intravenous infusion includes ala-TFPI at up to about 0.6 mg/ml, arginine hydrochloride at up to 300 mM, and sodium citrate buffer at pH 5.0-6.0. Certain solutes such as arginine, NaCl, sucrose, and mannitol serve to solubilize and/or stabilize ala-TFPI. See WO 96/40784.

Pharmaceutical formulations can include, for example, 0.01 to 1.0 mg/ml, 0.01 to 0.8 mg/ml, 0.01 to 0.5 mg/ml, 0.01 to 0.3 mg/ml, 0.01 to 0.2 mg/ml, or 0.01 to 0.1 mg/ml ala-TFPI; 150-450 mM, 150-400 mM, 150-350 mM, or 150-300 mM L-arginine; 0.1-50 mM, 0.1-40 mM, 0.1-30 mM, 0.1-25 mM, 0.1-15 mM, 0.1-10 mM, or 0.1-5 mM L-methionine; 5-50 mM, 5-45 mM, 5-40 mM, 5-35 mM, 5-30 mM, 5-25 mM, or 5-20 mM sodium citrate buffer. The pH of the formulations can range from 5.0-6, 5.0-5.8, 5.0-5.7, 5.0-5.6, or 5.0-5.5. Preferred pharmaceutical formulations include 0.15 mg/ml (±15, 10, or 5%) or 0.45 mg/ml (±15, 10, or 5%) recombinant ala-TFPI in 300 mM (±15, 10, or 5%) L-arginine, 5 mM (±15, 10, or 5%) L-methionine, 20 mM (±15, 10, or 5%) sodium citrate buffer, pH 5.5 (±15, 10, or 5%), with an osmolarity 560+/−110 mOsm/kg (±15, 10, or 5%).

An especially preferred formulation for intravenous infusion contains about 0.15 mg/ml ala-TFPI, 300 mM arginine hydrochloride, and 20 mM sodium citrate at pH 5.5. TFPI and TFPI analogs also can be formulated at concentrations up to about 0.15 mg/ml in 150 mM NaCl and 20 mM sodium phosphate or another buffer at pH 5.5-7.2, optionally with 0.005% or 0.01% (w/v) polysorbate 80 (Tween 80). Other formulations contain up to about 0.5 mg/ml TFPI, or TFPI analog in 10 mM sodium acetate at pH 5.5 containing either 150 mM NaCl, 8% (w/v) sucrose, or 4.5% (w/v) mannitol. TFPI and TFPI analogs can also be formulated at higher concentrations up to several mg/ml using high salt. For example, one formulation contains up to about 6.7 mg/ml ala-TFPI in 500 mM NaCl and 20 mM sodium phosphate at pH 7.0.

Further examples of formulants for TFPI and TFPI analogs include oils, polymers, vitamins, carbohydrates, amino acids, salts, buffers, albumin, surfactants, or bulking agents. Preferably carbohydrates include sugar or sugar alcohols such as mono, di, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcelloluose, or mixtures thereof. Sucrose is most preferred. Sugar alcohol is defined as a C₄ to C₈ hydrocarbon having an —OH group and includes galactitol, inositol, mannitol, xylitol, sorbitol, glycerol, and arabitol. Mannitol is most preferred. These sugars or sugar alcohols mentioned above may be used individually or in combination. There is no fixed limit to the amount used as long as the sugar or sugar alcohol is soluble in the aqueous preparation. Preferably, the sugar or sugar alcohol concentration is between 1.0 w/v % and 7.0 w/v %, more preferable between 2.0 and 6.0 w/v %.

Preferably amino acids include levorotary (L) forms of carnitine, arginine, and betaine; however, other amino acids may be added. It is also preferred to use a buffer in the composition to minimize pH changes in the solution before lyophilization or after reconstitution. Most any physiological buffer may be used, but citrate, phosphate, succinate, and glutamate buffers or mixtures thereof are preferred. Preferably, the concentration of the buffer is from 0.01 to 0.3 molar. Surfactants that can be added to the formulation are shown in EP Nos. 270,799 and 268,110.

After a liquid pharmaceutical composition of TFPI or a TFPI analog is prepared, it can be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) that may include additional ingredients. Upon reconstitution, the composition is preferably administered to subjects by continuous intravenous infusion.

### Dosages of TFPI and TFPI Analogs

TFPI or TFPI analogs are administered at a concentration that is therapeutically effective to treat and prevent severe bacterial infections, including severe pneumonia. Such doses also are effective for other acute or chronic inflammations, and generally diseases in which cytokines upregulate tissue factor expression. To accomplish this goal, TFPI or TFPI analogs preferably are administered intravenously. Methods to accomplish this administration are known to those of ordinary skill in the art. Generally, TFPI or TFPI analogs are preferably given at a dose between 1 mg/kg and 20 mg/kg, more preferably between 2 mg/kg and 15 mg/kg, most preferably between 2 and 10 mg/kg.

The above dosages are generally administered over a period of at least about 1 day, and usually several days, such that the total daily dose administered to a host in single or divided doses may be in amounts, for example, from about 2 to about 15 mg/kg body weight daily and preferably from about 4 to about 10 mg/kg. Dosage unit compositions may contain such amounts or submultiples thereof to make up the daily dose. Lower daily dosage amounts may be useful for prophylactic or other purposes, for example, from 1 μg/kg to 2 mg/kg. The amount of active ingredient that may be combined with the carrier materials to produce a single dosage form will vary depending upon the patient treated and the particular mode of administration.

The dosage regimen is selected in accordance with a variety of factors, including the type, age, weight, sex, diet and medical condition of the patient, the severity of the condition, the route of administration, pharmacological considerations such as the activity, efficacy, pharmacokinetic and toxicology profiles, whether a drug delivery system is utilized and whether the compound is administered as part of a drug combination. Thus, the dosage regimen actually employed may vary widely and therefore may deviate from the preferred dosage regimen set forth above. Preferably, doses of TFPI or TFPI analogs should not exceed a dose rate equivalent to a dose rate of ala-TFPI of about 0.66 mg/kg/hr. In addition to dose rate, the duration of infusion of TFPI or a TFPI analog will depend on the clinical severity of each individual patient, and determination of the appropriate period is well within the skill of the ordinary clinician. Infusion can be carried out, for example, for between 10-200 hours, 10-150 hours, 24-120 hours, 36-100 hours or 24-96 hours. One or more courses of treatment, typically two, can be carried out, depending on the judgment of the treating physician.

### TFPI Dose Administration

When TFPI or a TFPI analog is given at a dose rate equivalent to administration of ala-TFPI at a dose rate of at least about 0.00025 mg/kg/hr (0.00417 μg/kg/min) and less than about 0.50 mg/kg/hr (8.33 μg/kg/min), efficacy in treating severe bacterial infections is retained and adverse side effects, such as bleeding, are minimized. For improved combined efficacy and safety, the dose rate preferably is equivalent to a dose rate of ala-TFPI of at least about 0.010 mg/kg/hr (0.167 μg/kg/min) and less than about 0.1 mg/kg/hr (1.67 μg/kg/min), or equivalent to a dose rate of ala-TFPI of at least about 0.020 mg/kg/hr and less than about 0.080 mg/kg/hr, and most preferably equivalent to a dose rate of ala-TFPI of about 0.025 mg/kg/hr (0.417 μg/kg/min) to about 0.075 mg/kg/hr (1.251 μg/kg/min). The route of administration is generally by intravenous administration, with continuous intravenous infusion preferred. Infusion can be administered for at least about 72, 96, 100, 120, or 240 hours. Preferably, continuous infusion is administered for 3 to 8 days, more preferably 3 to 6 days, and most preferably for about 4 days.

To administer “by continuous infusion” means that the infusion is maintained at approximately the prescribed rate without substantial interruption for most of the prescribed duration. Alternatively, intermittent intravenous infusion can be used. If intermittent infusion is used, then a time-averaged dose rate should be used which is equivalent to the dose rates described above for continuous infusion. In addition, the program of intermittent infusion must result in a maximum serum concentration not more than about 20% above the maximum concentration obtained using continuous infusion. To avoid adverse reactions in the patient, particularly side effects involving bleeding, the dose rate should be less than a dose rate that is equivalent to continuous intravenous infusion of ala-TFPI at about 0.1 mg/kg/hr.

All doses described herein, including dose rates and total doses, are subject to up to 10% variation in practice due to errors in determining protein concentration and biological activity with the prothrombin assay. Thus, any actually administered dose up to 10% higher or 10% lower than a dose stated herein is considered to be equivalent to the stated dose. For this reason, all doses have been stated as “about” a specific dose. For example, a dose described as “about 0.025 mg/kg/hr” is considered equivalent to any actual dose ranging from 0.0225 to 0.0275 mg/kg/hr.

Preferred dosage regimens include two intravenous doses of 100 ml each of 0.15 mg/ml or 0.45 mg/ml recombinant ala-TFPI in 300 mM L-arginine, 5 mM L-methionine, 20 mM sodium citrate buffer, ph 5.5, with an osmolarity 560+/−110 mOsm/kg.

A bolus injection or a briefly higher infusion rate of TFPI or an analog of TFPI may also be employed in the practice of the present invention if followed by low dose TFPI administration. For example, a bolus injection or higher infusion rate can be used to reduce the equilibration time of administered TFPI or TFPI analog in the circulation of a patient. In doing so, the eventual steady state plasma level of TFPI can be reached more rapidly and receptors for TFPI can be saturated faster. Administration of ala-TFPI to humans at about 0.025 mg/kg/hr for 2 hours increases plasma levels of TFPI (plus ala-TFPI) from about 80 ng/ml to about 125 ng/ml, or an increase of approximately 50%. The same level will be reached faster if the infusion rate is increased, or a bolus injection is used. Higher infusion rates will result in higher levels if infusion is continued until steady state is obtained. Steady state level for administration of ala-TFPI at about 0.050 mg/kg/hr was found to be about 300 ng/ml, and for administration of ala-TFPI at about 0.33 or about 0.66 mg/kg/hr was found to be about at least 2 μg/ml in patients suffering from severe bacterial infections.

Total daily dose administered to a host in a single continuous infusion or in divided infusion doses may be in amounts, for example, equivalent to administration of at least about 0.006 mg/kg/day to less than about 1.2 mg/kg/day of ala-TFPI, more usually equivalent to administration of from about 0.24 mg/kg/day to less than about 1.2 mg/kg/day of ala-TFPI, and preferably equivalent to about 0.6 mg/kg/day of ala-TFPI. Lower amounts within this range may be useful for prophylactic or other purposes. The dosing protocols of the invention can also be expressed as the total dose administered to the patient. The total dose is the mathematical product of the rate of infusion and the total time of infusion. For example, at the preferred dose rate of about 0.025 mg/kg/hr for ala-TFPI and the preferred infusion time of 96 hours, the total dose is about 2.4 mg ala-TFPI per kg body weight. The total dose of TFPI administered according to the invention is equivalent to at least about 0.75 μg/kg and less than about 4.8 mg/kg of ala-TFPI. Preferably the total dose is equivalent to at least about 1 mg/kg and less than about 4.8 mg/kg of ala-TFPI. More preferably the total dose is equivalent to about 2.4 mg/kg of ala-TFPI.

The dosing regimens described above, including dosing rate on a mg/kg/hr basis and total daily dose, are expressed as a dose “equivalent to administration of reference ala-TFPI.” This means that they are determined quantitatively by normalization to a dose of “reference ala-TFPI” which is defined as mature, 100% pure (on a protein basis), properly folded, biologically active, non-glycosylated ala-TFPI. Ala-TFPI is an analog of TFPI whose amino acid sequence is depicted in SEQ ID NO:2. Other forms of TFPI can also be used in the invention, including mature, full-length TFPI and analogs thereof. To determine the appropriate dosing range for practicing the invention with forms of TFPI other than ala-TFPI and with preparations of ala-TFPI or another TFPI analog that are less than 100% pure, the dosing ranges described herein for reference ala-TFPI can be adjusted based on the intrinsic biological activity of the particular form of TFPI and further adjusted based on the biochemical purity of the preparation.

The intrinsic biological activity of TFPI or a TFPI analog refers to the specific activity, as defined by the prothrombin assay, of the mature, 100% pure, properly folded TFPI or TFPI analog. Thus, the equivalent dose is calculated as (reference ala-TFPI dose)/((relative intrinsic activity)×(biochemical purity)), where relative intrinsic activity refers to (intrinsic activity of analog)/(intrinsic activity of reference ala-TFPI). For example, if a particular TFPI analog has an intrinsic biological activity which is 80% that of reference ala-TFPI, then the equivalent dose for the particular TFPI analog are obtained by dividing the dose values for reference ala-TFPI by 0.8. Further, if the formulation administered to a patient is, for example, only 90% biochemically pure, i.e., comprising 10% of molecular species which lack biological activity of TFPI, then an additional correction of the reference dose values for ala-TFPI is performed by dividing the dose values by 0.9. Thus, for a hypothetical TFPI analog which has 80% of the intrinsic activity of ala-TFPI and is 90% biochemically pure as administered, a dose rate equivalent to administration of reference ala-TFPI at 0.025 mg/kg/hr would be 0.0347 mg/kg/hr (i.e., 0.025/(0.8×0.9)).

Equivalent doses can also be determined without knowing either intrinsic activity or biochemical purity by determining relative biological activity. Relative biological activity can be determined by comparing a particular TFPI analog to a TFPI biological activity standard using the prothrombin time assay. For example, ala-TFPI produced according to the method of Example 9 of WO 96/40784, which contains about 85% biologically active TFPI molecular species, can be used as a TFPI biological activity standard. Ala-TFPI produced according to the method of Example 9 of WO 96/40784 has about 85% of the activity of reference ala-TFPI in the prothrombin assay. In plotting a prothrombin time standard curve, the log of clotting time is plotted against the log of TFPI concentration. If the TFPI biological activity standard possesses 85% of the activity of reference ala-TFPI, then a standard curve can be prepared which is equivalent to that for reference ala-TFPI if the concentrations of the TFPI biological activity standard are multiplied by 0.85 prior to plotting, so that the activity plotted is equivalent to the activity of 100% pure reference ala-TFPI. When the clotting time for a particular TFPI analog is compared to the standard curve, the equivalent concentration of reference ala-TFPI can be read off the curve.

Alternatively, if the slope of the linear portion of the standard curve is obtained by linear regression analysis, then the slope can be corrected based on the activity of the TFPI biological activity standard relative to reference ala-TFPI. The relative biological activity of a particular TFPI analog is thus equal to the ratio of reference ala-TFPI activity to the activity of the analog. For example, if a particular analog requires 1.43 μg to produce the same prothrombin time activity as 1.00 μg of reference ala-TFPI, then the relative biological activity of the analog is 1.00/1.43, or 0.7. For that analog, the equivalent dose to a reference ala-TFPI dose is obtained by dividing the reference ala-TFPI dose by the relative biological activity of the analog. For example, a 0.025 mg/kg/hr dose for reference ala-TFPI would be equivalent to 0.0357 mg/kg/hr of the analog (i.e., 0.025/0.7).

In some embodiments TFPI or TFPI analogs are administered to a patient who has had previous heparin treatment. In this case, the patient preferably has not received heparin for at least 8 hours before administration of TFPI or a TFPI analog. Treatment with unfractionated heparin may require a longer “washout” period, e.g., 20, 21, 22, 23, or 24 hours. Typical washout periods for patients treated with low molecular weight heparin are 9, 10, 11, or 12 hours.

While TFPI or a TFPI analog can be administered as the sole active anticoagulation pharmaceutical agent, these molecules also can be used in combination with one or more additional therapeutic agents to provide a combination therapy for the treatment of sever pneumonia. Such additional therapeutic agents include antibodies such as, for example, anti-endotoxin, monoclonal antibodies (e.g., endotoxin-binding Mabs) and anti-TNF products such as an anti-TNF murine Mab. TFPI and TFPI analogs can also be combined with interleukin-1 receptor antagonists, bactericidal/permeability increasing (BPI) protein, immunostimulant, compounds having anti-inflammatory activity such as PAF antagonists, and cell adhesion blockers (e.g., antiplatelet agents such as GPIIb/IIIa inhibitors).

Patients with APACHE II scores lower than 38 but above 25 (e.g., between 25 and 37, or 25, 26, 27, 28, 29, 30, 31, 32, 33, 34, 35, 36, or 37) can additionally be treated with activated protein C (e.g., XIGRIS®). Activated protein C typically is administered at an infusion rate of 24 μg/kg/hr for a total of 96 hours. Adjustment of this dosage regimen can be adjusted by the skilled physician according to an individual patient's clinical presentation.

When administered as a combination, the therapeutic agents can be formulated as separate compositions that are given at the same time or different times. Preferably, additional therapeutic agents are given either at the same time (i.e., during the administration period of TFPI or TFPI analogs) or within 24 hours of the administration period of TFPI or TFPI analogs (i.e., within 24 hours prior to the start of, or within 24 hours after the end of, the administration period of TFPI or TFPI analogs). Additional therapeutic agents can also be given as a single composition together with the TFPI or TFPI analogs.

TFPI or a TFPI analog also can be given in combination with other agents that would be effective to treat severe bacterial infections, particularly pneumonia. For example, the following may be administered in combination with TFPI or a TFPI analog: antibiotics that can treat the underlying bacterial infection, monoclonal antibodies that are directed against bacterial cell wall components, receptors that can complex with cytokines that are involved in the severe pneumonia pathway, and generally any agent or protein that can interact with cytokines or other activated or amplified physiological pathways including complement proteins to attenuate severe pneumonia and/or its symptoms.

Useful antibiotics include those in the general category of: beta-lactam rings (penicillin), amino sugars in glycosidic linkage (aminoglycosides), macrocyclic lactone rings (macrolides), polycyclic derivatives of napthacenecarboxanide (tetracyclines), nitrobenzene derivatives of dichloroacetic acid, peptides (bacitracin, gramicidin, and polymyxin), large rings with a conjugated double bond system (polyenes), sulfa drugs derived from sulfanilamide (sulfonamides), 5-nitro-2-furanyl groups (nitrofurans), quinolone carboxylic acids (nalidixic acid), and many others. Other antibiotics and more versions of the above specific antibiotics may be found in Encyclopedia of Chemical Technology, 3rd Edition, Kirk-Othymer (ed.), Vol. 2, pages 782-1036 (1978) and Vol. 3, pages 1-78, Zinsser, MicroBiology, 17th Edition W. Joldik et al. (Eds.) pages 235-277 (1980), or Dorland's Illustrated Medical Dictionary, 27th Edition, W.B. Saunders Company (1988).

Other agents that may be combined with TFPI or a TFPI analog include endotoxin antagonists such as E5531 (a Lipid A analog, see Asai et al., Biol. Pharm. Bull. 22:432 (1999)), TF analogs with anticoagulant. activity (see, e.g., Kelley et al., Blood 89:3219 (1997) and Lee & Kelley, J. Biol. Chem. 273:4149 (1998)), monoclonal antibodies directed to cytokines, such as those monoclonal antibodies directed to IL-6 or M-CSF, see U.S. Ser. No. 07/451,218, filed Dec. 15, 1989, and monoclonal antibodies directed to TNF (see Cerami et al., U.S. Pat. No. 4,603,106), inhibitors of protein that cleave the mature TNF prohormone from the cell in which it was produced (see U.S. Ser. No. 07/395,253, filed Aug. 16, 1989), antagonists of IL-1 (see U.S. Ser. No. 07/517,276, filed May 1, 1990), inhibitors of IL-6 cytokine expression such as inhibin (see U.S. Pat. No. 5,942,220), and receptor based inhibitors of various cytokines such as IL-1. Antibodies to complement or protein inhibitors of complement, such as CR₁, DAF, and MCP also can be used.

All patents, patent applications, and references cited in this disclosure are incorporated herein by reference in their entireties.

The present invention will now be illustrated by reference to the following examples that set forth particularly advantageous embodiments. However, it should be noted that these embodiments are illustrative and are not to be construed as restricting the invention in any way.

### EXAMPLE 1

### Materials

Monoclonal antibodies to Kunitz domain 1 (4904) or Kunitz domain 2 (4903) of TFPI were purchased from American Diagnostica Inc. (Greenwich, Conn.). We made additional monoclonal antibodies (2H8, against Kunitz domain 1, and 17, against the C-terminus). The control mouse IgG1 was purchased from Jackson ImmunoResearch Lab Inc. Human umbilical vein endothelial cells (HUVEC) were purchased from Clonetics (San Diego, Calif.) and cultured at 37° C. in EBM-2 media (Clonetics). Lipopolysaccharide from E. coli (LPS) was purchased from Sigma (L-2654).

In the examples that follow, all exogenous TFPI is ala-TFPI.

### EXAMPLE 2

### Whole Blood Assay

Blood was drawn from healthy donors in the absence of anticoagulants and under conditions which minimized platelet activation. Donor selection excluded use of aspirin or other blood thinners, cholesterol-lowering drugs, anti-inflammatory agents, anti-histamines, and antibiotics. The reagent mixtures containing 10 ng/ml LPS and different concentrations of the mAbs with or without ala-TFPI were pre-assembled in 96-well dishes. Blood was added to 1:10 ratio in RPMI-1640 medium with or without 10% FBS of a total volume of 200 μl within 10 min of donation. The diluted blood samples were incubated for 16 to 20 hrs at 37° C. in a cell culture incubator, and aliquots of the cell supernatant were analyzed for secretion of inflammatory cytokines using an IL-6 ELISA kit (Biosource). Release of other cytokines was measured using a LINCOPLEX® cytokine kit (LINCO Research).

### EXAMPLE 3

### Whole Blood Plus HUVEC Assay

HUVEC cells were plated at 5000-7000 cells/96 well in EBM-2 media with 10 ng/ml LPS a day before the experiment. On the day of the experiment, the medium was removed and replaced with a pre-assembled reagent mix containing 10 ng/ml LPS and different concentrations of the mAbs with or without ala-TFPI in RPMI-1640 medium with or without 10% FBS. Then the blood was added at a ratio of 1:10. The whole blood assay was carried out as described above.

### EXAMPLE 4

### Patient Baseline TFPI Correlates with Mortality

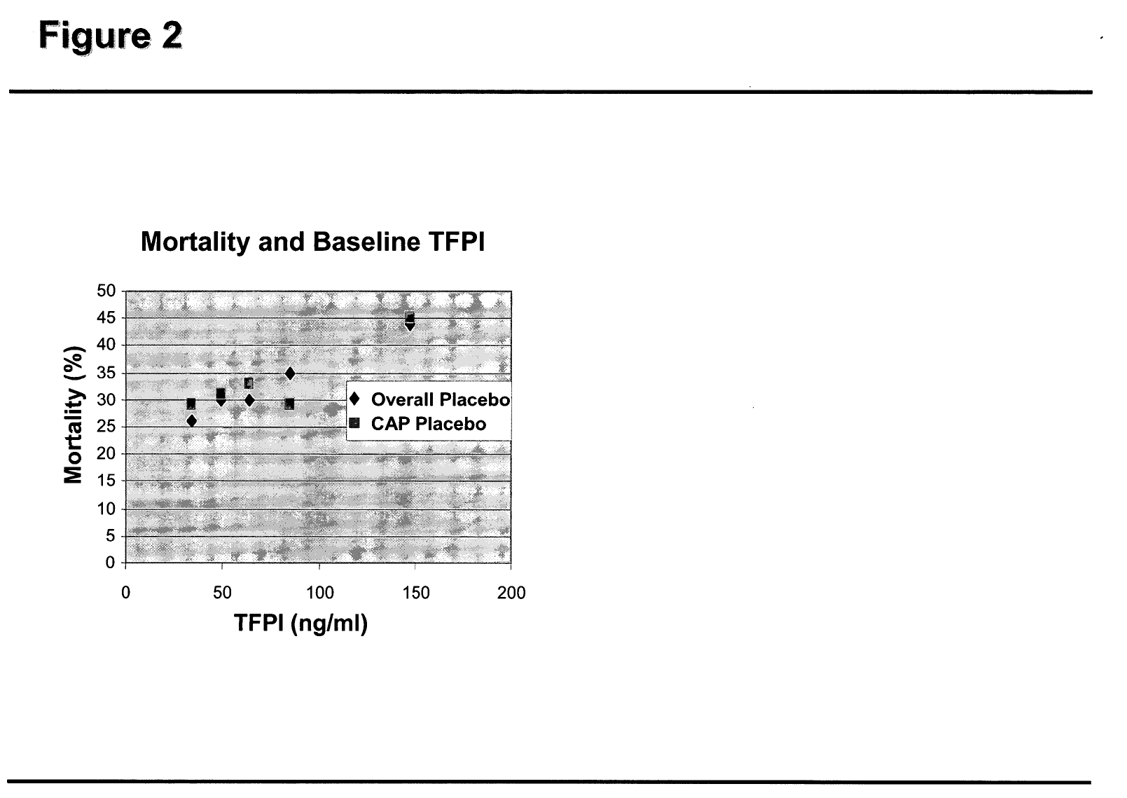

In a large Phase III clinical trial for treating severe sepsis of multiple causes with ala-TFPI, we found large subsets of patients that apparently benefited from ala-TFPI treatment. In particular we noted that patients with CAP performed well. Among the analyses run in this trial we measured baseline plasma concentration of TFPI by ELISA. As shown in FIG. 2, patient baseline TFPI correlated with mortality. This was true either overall or in the CAP subset. This result suggests that, as sepsis worsens, TFPI is inactivated and released from blood cells. Exogenous ala-TFPI apparently replaces endogenous TFPI lost during infection.

### EXAMPLE 5

### Simulation of TFPI Turnover and Induction of TFPI Degradation in a Whole Blood Sepsis Model

We created a model of septic blood by adding serum to normal blood. This causes rapid coagulation and induces a lipopolysacchide (LPS) dependent secretion of many cytokines, creating a condition similar to what is observed in blood from septic patients.

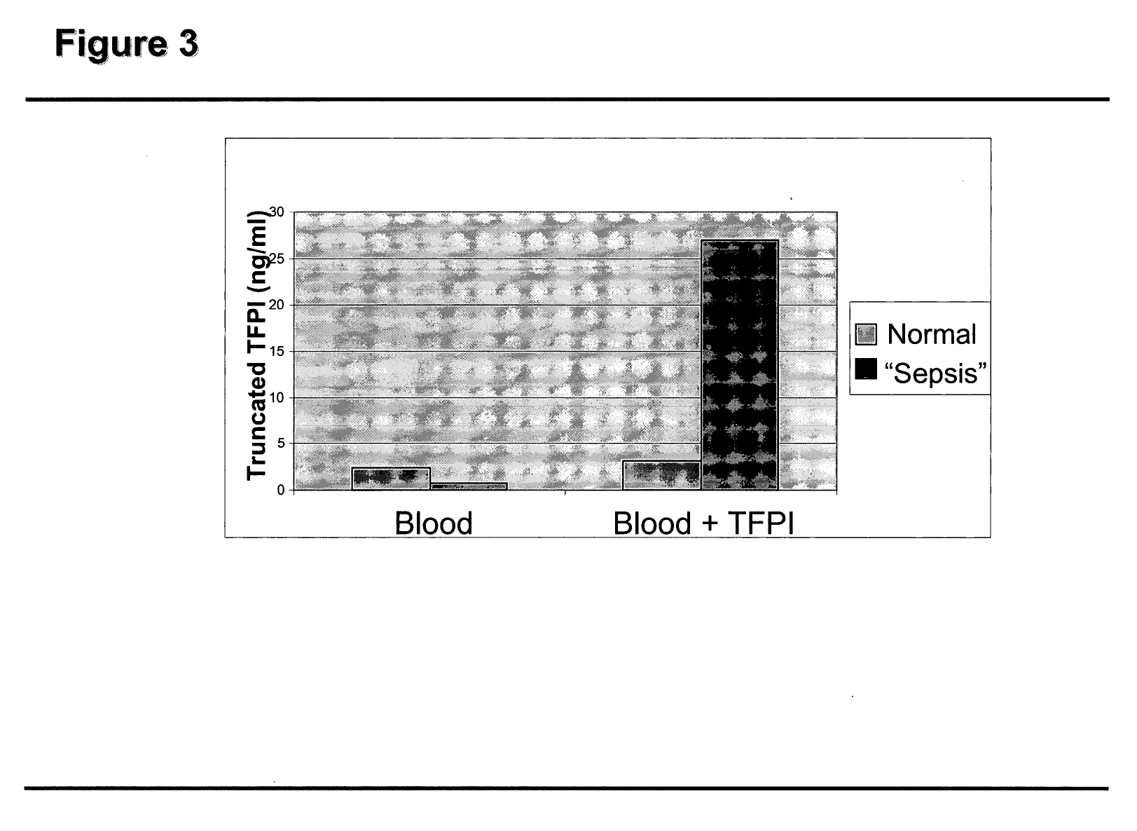

When ala-TFPI was added to whole blood it remained intact at the C-terminus. In our septic model, however, the C-terminal domain was cleaved from added ala-TFPI. The results are shown in FIG. 3. This data indicates that conditions which activate coagulation and induce inflammatory cytokines in blood lead to removal of the C-terminal domain of TFPI. Up to 95% of normal plasma TFPI has a C-terminal deletion. C-terminally truncated TFPI has little activity in blood assays.

### EXAMPLE 6

### Affect of Added ala-TFPI on the Survival of Patients with Various Endogenous TFPI Levels

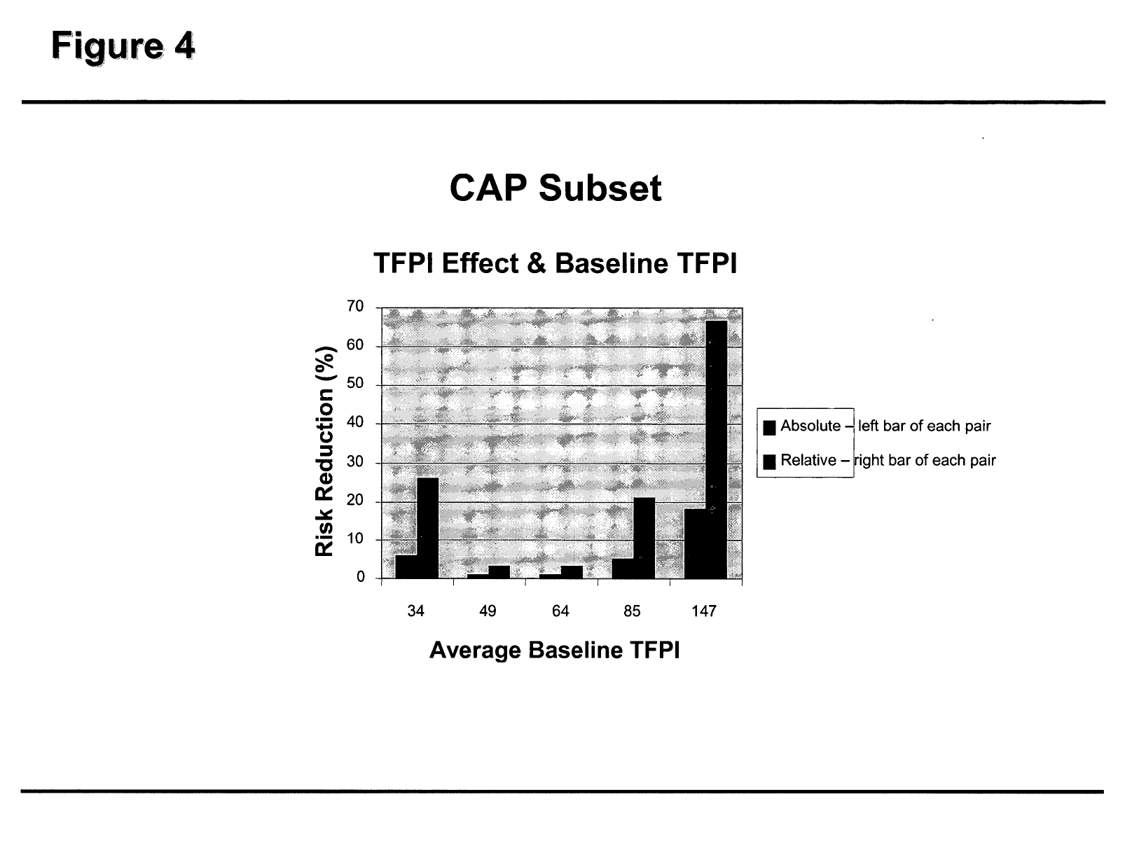

Added ala-TFPI appeared to be effective in patients in whom baseline TFPI was abnormal (FIG. 4). When CAP patients were separated into subsets by baseline TFPI levels, we found that ala-TFPI was effective in patients with below normal baseline TFPI and in patients with elevated baseline TFPI. The fact that added ala-TFPI improved the patients with elevated baseline TFPI is consistent with data which shows plasma TFPI is inactive and that the ala-TFPI drug is the fully active form.

### EXAMPLE 7

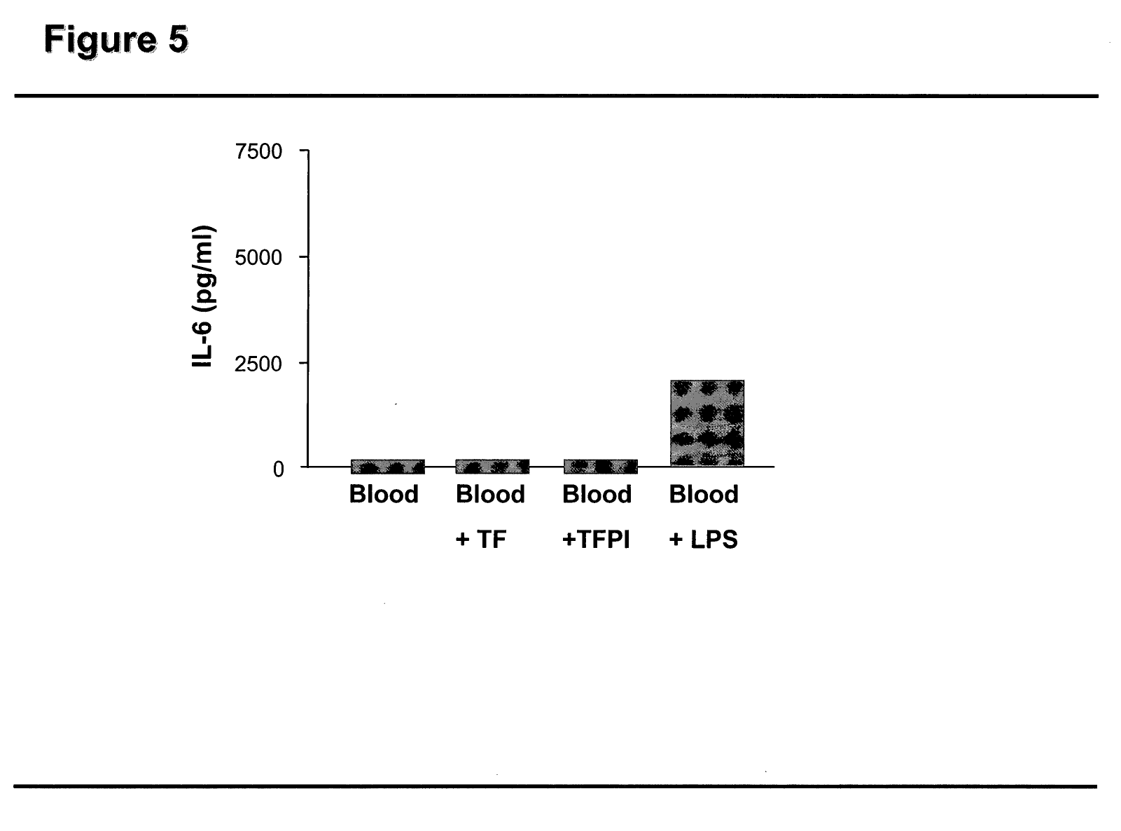

We explored TFPI activity in diluted whole blood from healthy donors. We see several activities consistent with TFPI being part of signaling complexes that regulate antibacterial activity through inflammatory cytokines. Inflammatory cytokines levels in normal blood were very low and remained low when exogenous ala-TFPI was added (FIG. 5). As expected, adding LPS to normal blood elevated inflammatory cytokines including IL-6, IL-1β and TNF-α. This reaction is sensitive to plasma TFPI concentration; any loss of functional TFPI will degrade the response.

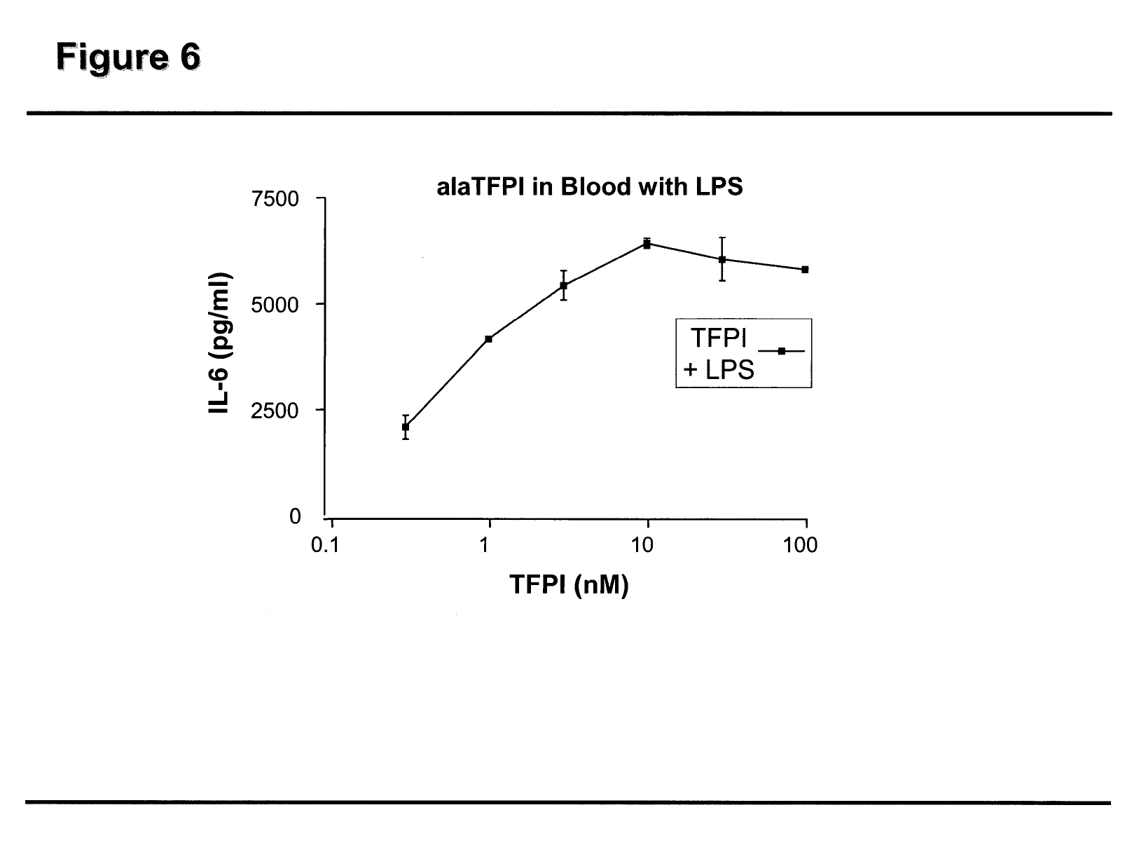

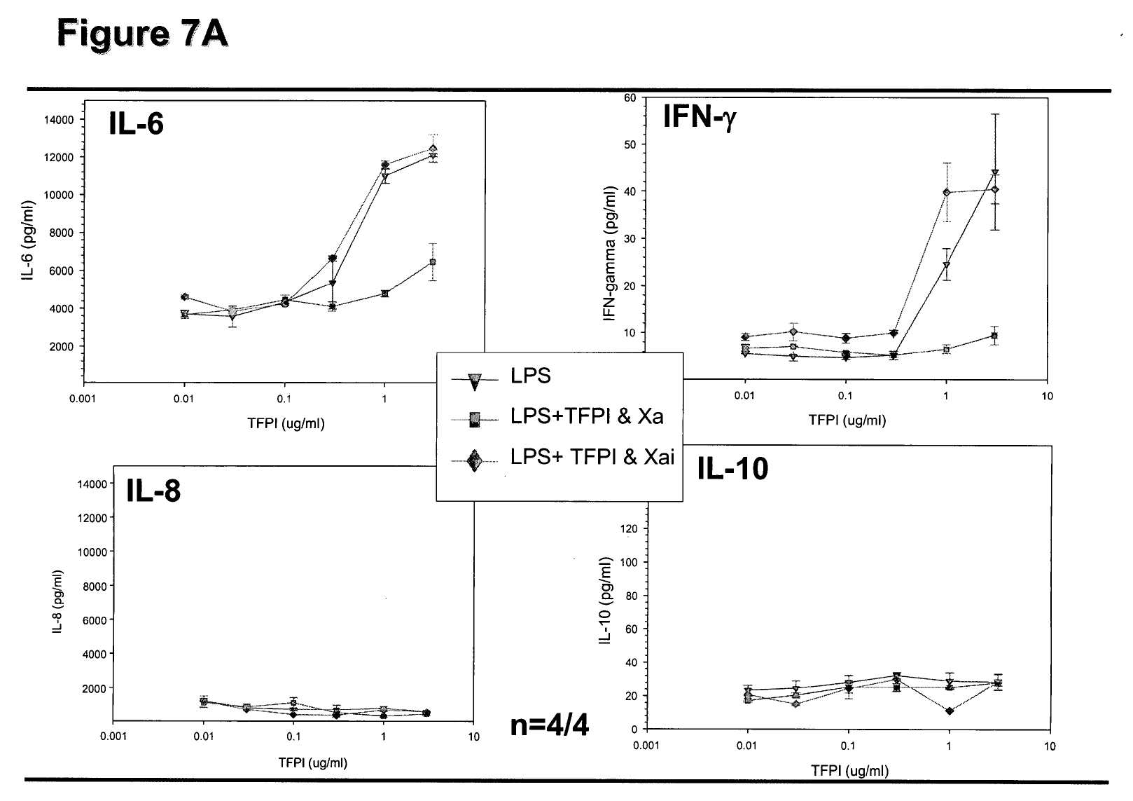

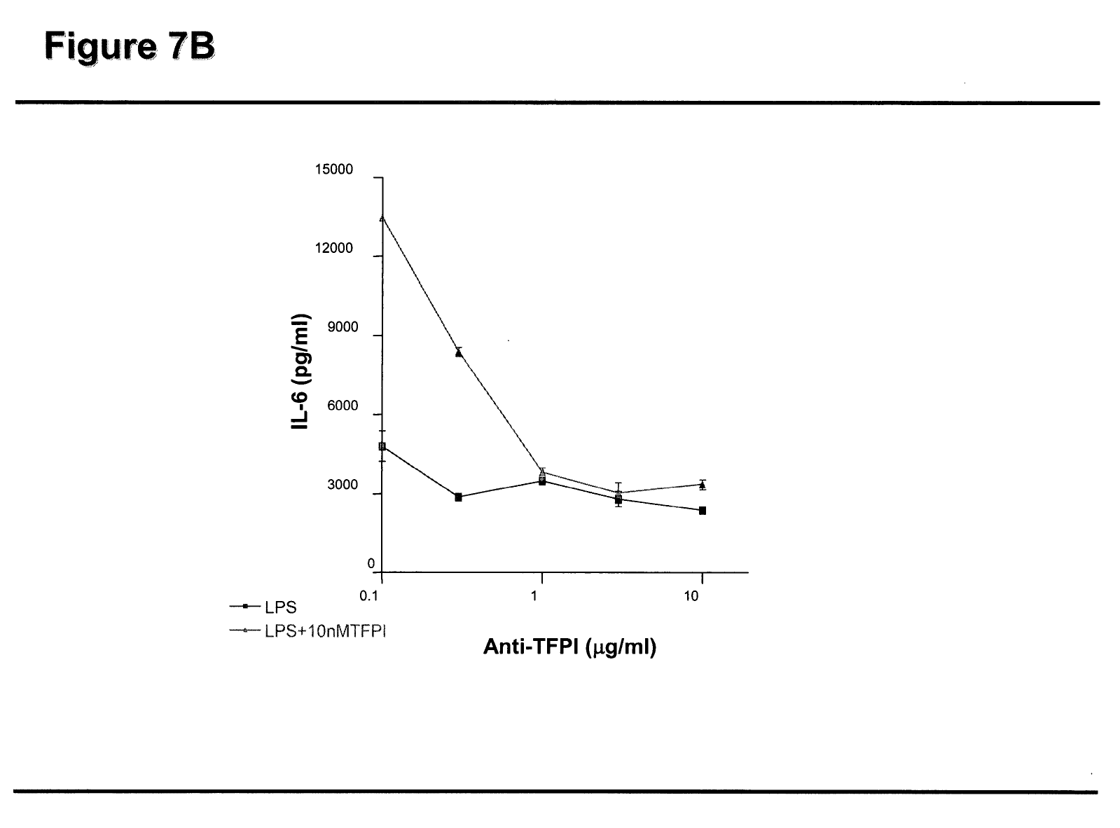

Unexpectedly, LPS also sensitized blood to TFPI such that added ala-TFPI induced interferon-γ (IFN-γ), IL-6, IL-1β, and TNF-α (FIGS. 6, 7A); the increases in IL-6, IFN-γ, TNF-α and IL1β are all reversed by small dose of anti-TFPI (FIG. 7B). This spectrum of cytokines is associated with activation of macrophages to increase their antibacterial activity.

In the current understanding of cellular immunology, cellular immunity is mediated by T_{H}1 cells. T_{H}1 cell activation requires antigen recognition and clonal expansion of the relevant T cells, a process taking a week. The ala-TFPI driven induction occurred after hours, suggesting that this form of activation could play an important role in the critical early phases of an infection before the adaptive immune system is fully engaged.

These results indicate that TFPI seems to be functioning as a ligand in a signaling cascade rather than a protease inhibitor in the coagulation cascade.

### EXAMPLE 8

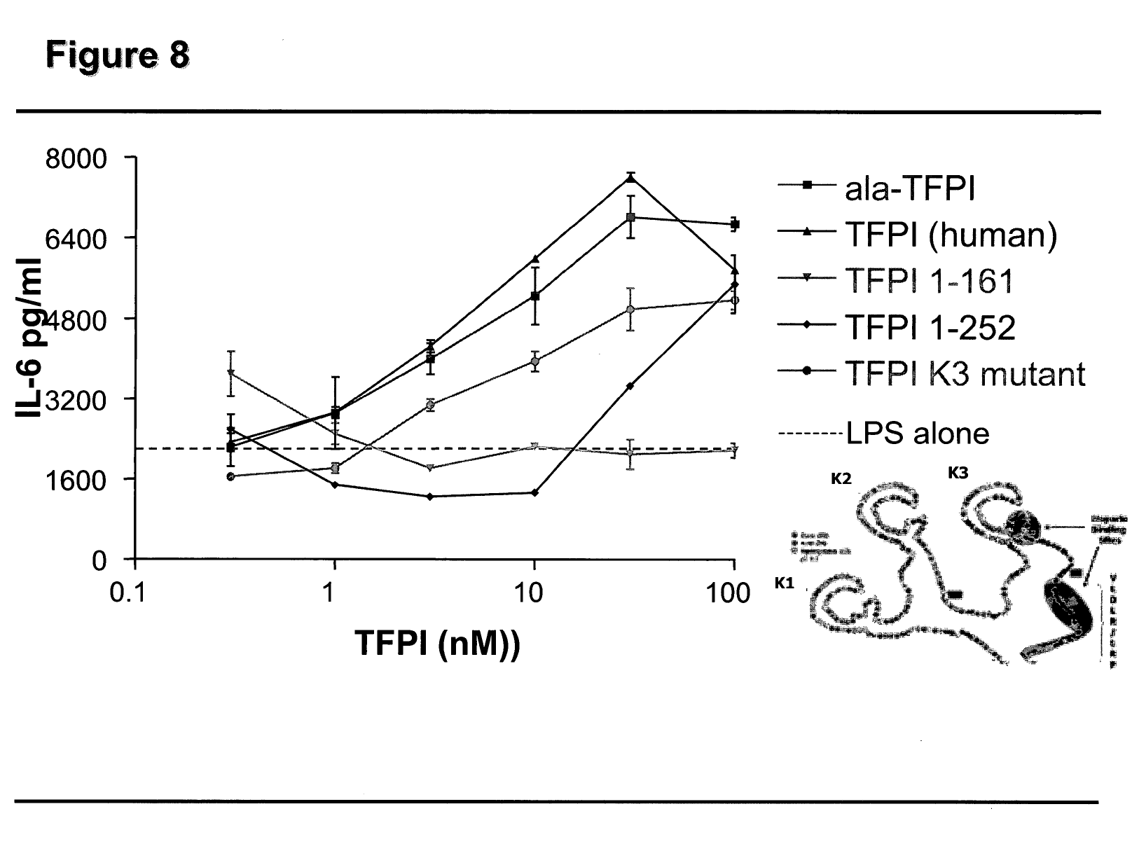

Recent literature points to Kunitz domain 3 (K3) as an essential domain for interaction of TFPI with its receptors, while K3 appears to be unimportant for inhibition of coagulation. Piro & Broze, Circulation. 2004 Dec. 7;110(23):3567-72. Using whole blood assays we mapped the domains of TFPI required for TFPI to induce cytokines. The results are shown in FIG. 8. We found that C-terminally deleted TFPI still had activity; while a deletion mutant lacking both the C-terminal domain and K3 was inactive. These data are consistent with the proposed signaling activity of TFPI being anchored to a receptor through K3.

### EXAMPLE 9

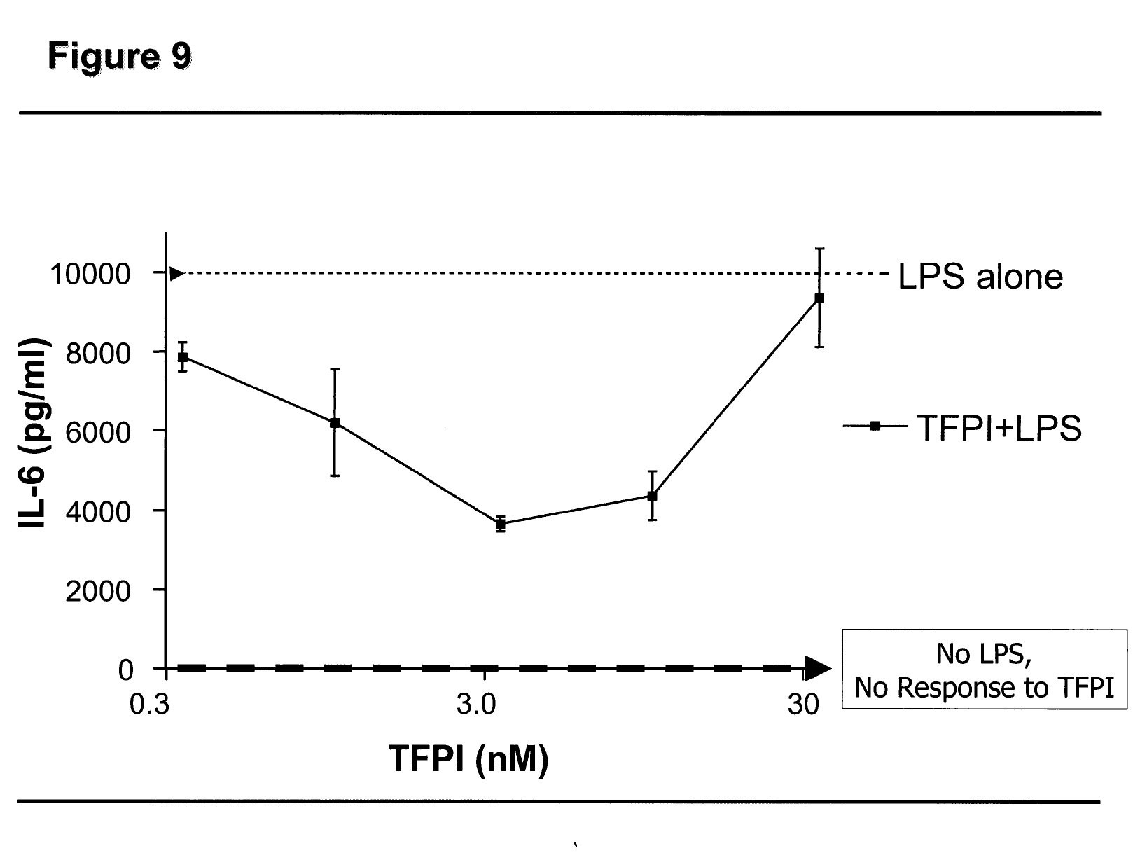

As noted above, adding serum to blood containing LPS induces a spectrum of cytokines; these include IL-1β, TNF-α, IL-6, IL-8, IL-10 but lack IFN-γ. This collection matches the output of activated macrophages. Exogenously added 3 to 10 nM ala-TFPI is able to inhibit these serum induced cytokines (FIGS. 7 and 9).

### EXAMPLE 10

### TFPI Inhibits IL-6 Production Driven by Factor Xa

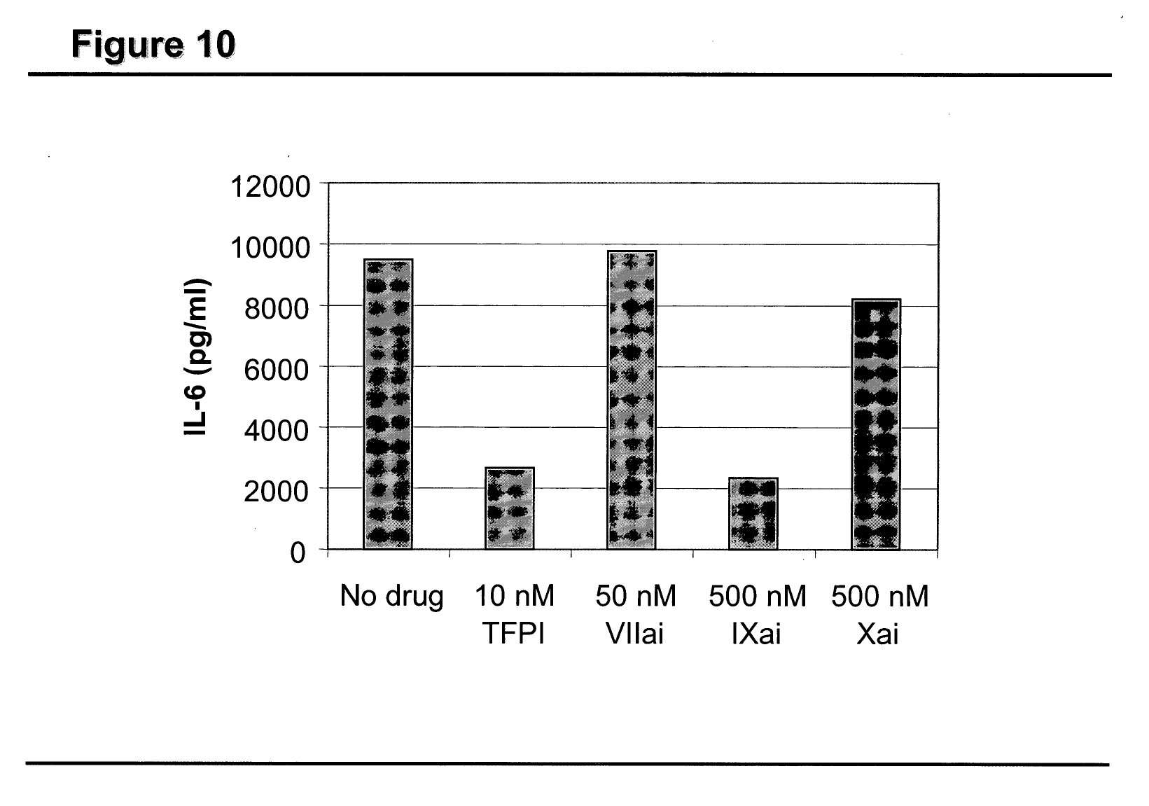

The inhibitory activity of TFPI on IL-6 production appears to be through TFPI's ability to inhibit Xa production. FIG. 10 shows the results of whole blood assays measuring IL-6 triggered by LPS and serum in the presence of various inhibitors (the inhibitors DEGR-VIIa, DEGR-IXa, and DEGR-Xa are written: VIIai, IXai and Xai). When we tested several inhibitors, we found that site-inactivated Vila had no activity, showing that the cytokines were not driven by tissue factor (TF)-mediated activation of clotting. Contrary to our expectations, site inactivated IXa was about as potent as TFPI, showing that the cytokine secretion involved formation of factor Xa driven through the intrinsic clotting cascade. According to the literature, Xa should induce cytokines through its ability to make thrombin. In contradiction to this, we found that site-inactivated Xa had no effect.

These results show that cytokines can be driven by Xa directly and that TFPI in this type of reaction is effective through its ability to inhibit Xa production.

### EXAMPLE 11

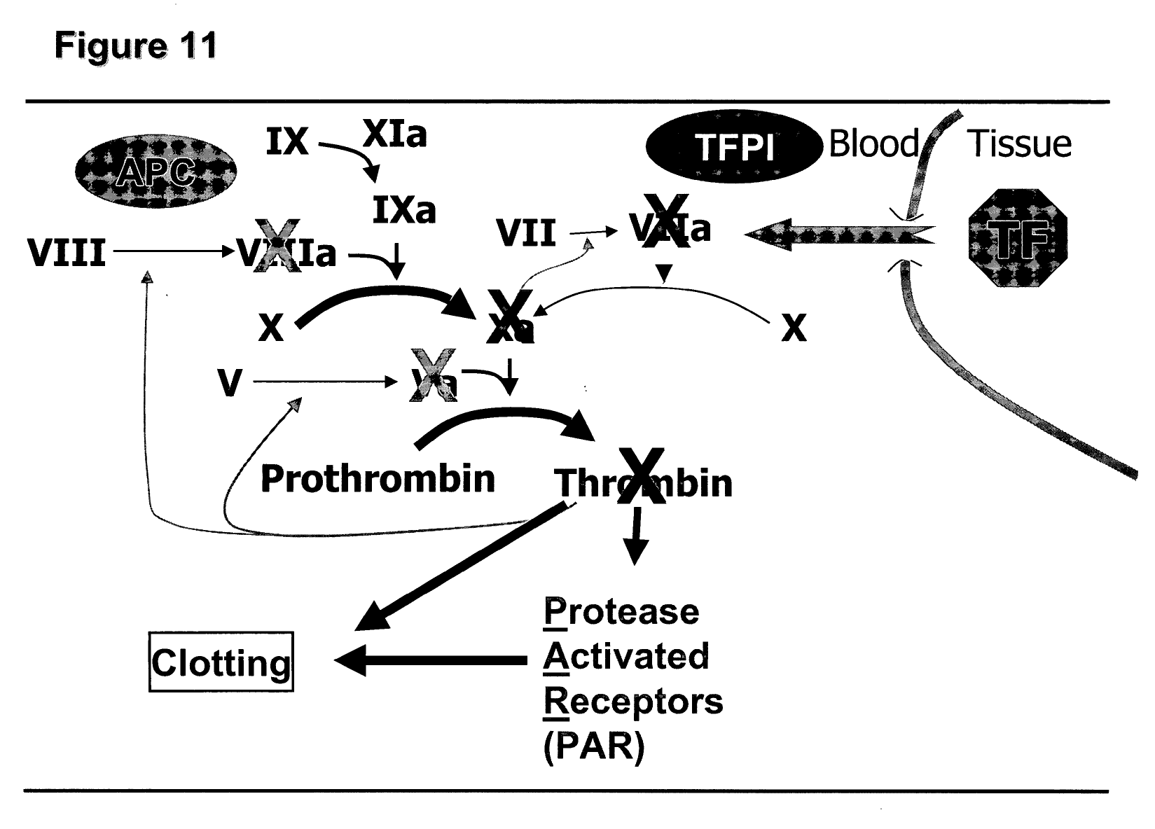

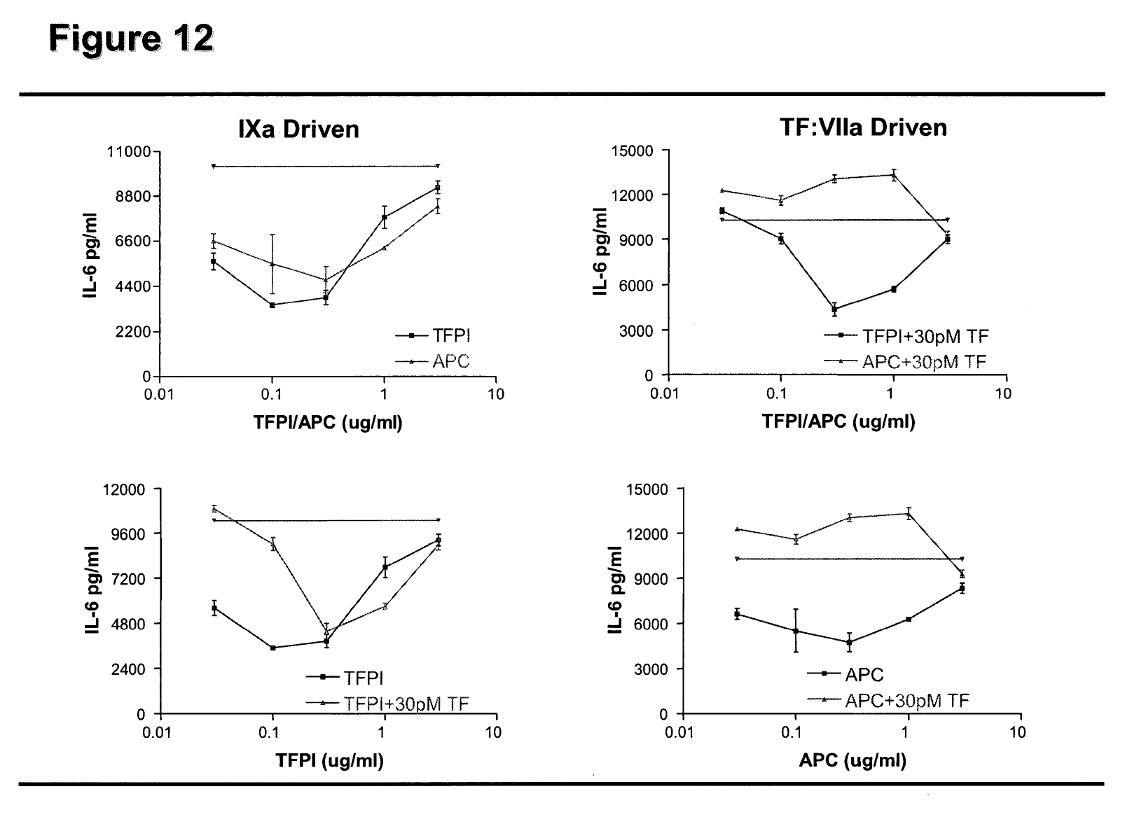

TFPI and APC (activated protein C) have very similar effects on coagulation because each limits the production of thrombin (FIG. 11). However, if cytokine induction is driven by factor Xa, then these drugs should have different effects on IL-6 induction. APC inhibits production of Xa by destroying factor VIIIa but has no effect on TF driven production of Xa. In our model of septic blood we observed exactly this result when we added a small amount of TF to the reaction (FIG. 12). In a reaction where IXa is driving IL-6 we find that both proteins can inhibit serum-driven inflammation as reflected in IL-6 levels. As expected, APC inhibited the production of IL-6 induced by the intrinsic pathway. In the presence of TF, however, only TFPI could inhibit the production of IL-6.

### EXAMPLE 12

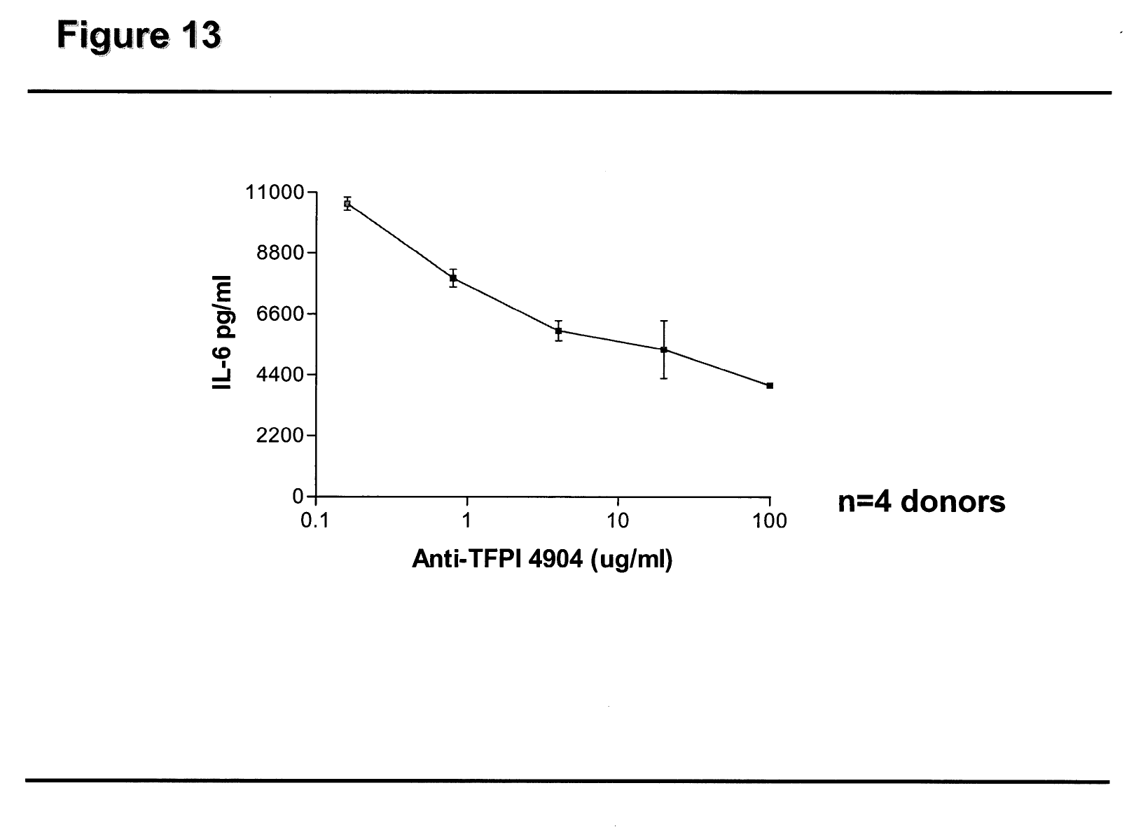

Blood cells are known to have TFPI on their surfaces. To determine the activity of this TFPI we inhibited its function with anti-TFPI antibodies and measured cytokines (FIG. 13). In an apparent contradiction to the inhibition seen with added ala-TFPI, anti-TFPI also inhibited IL-6 secretion. These data parallel the result of adding ala-TFPI to normal blood plus LPS. From these data we conclude that TFPI is part of a signaling complex.

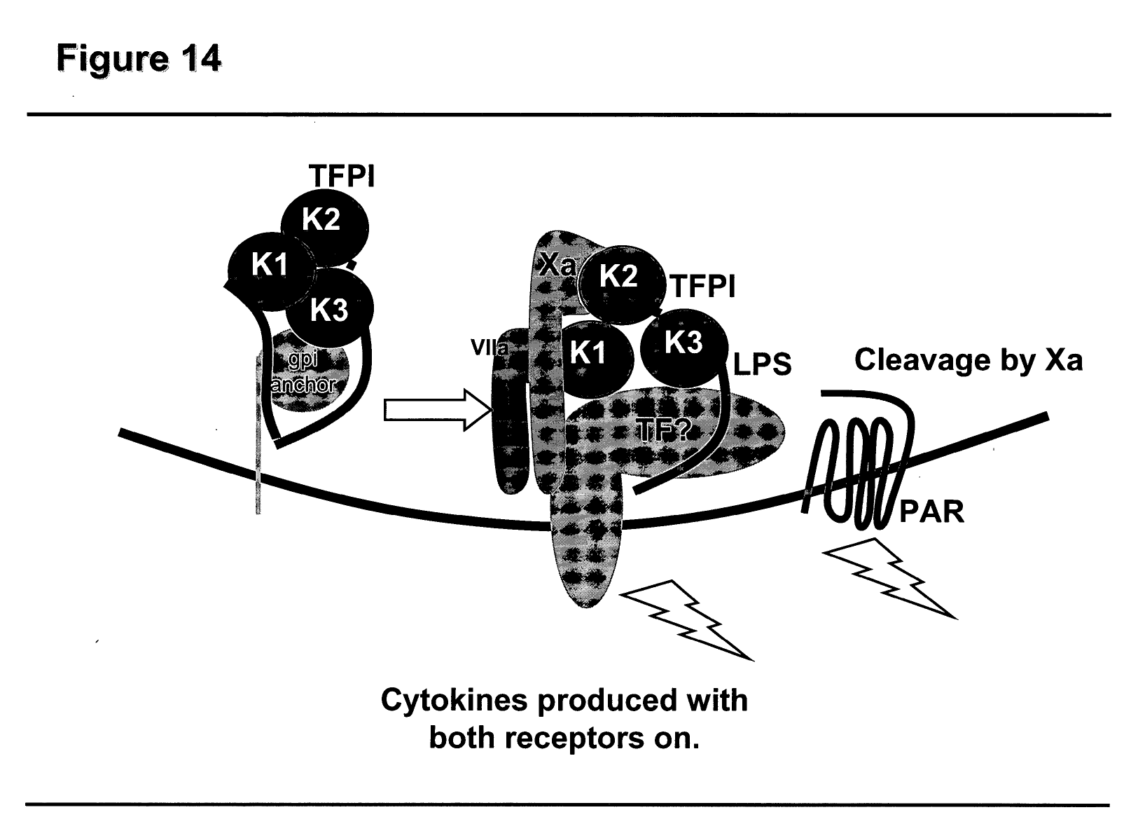

A model of the relocation of a TF:VIIa:Xa complex to caveolae, where it combines with TFPI, is shown in FIG. 14. This complex is dependent on Xa for its formation. While not wishing to be bound by this mechanism, we think the function of the complex is to activate cytokine secretion and enhance bacterial clearance mechanisms. Because formation of the complex is dependent on Xa, an excess of TFPI will prevent complex formation, as will a neutralizing antibody.

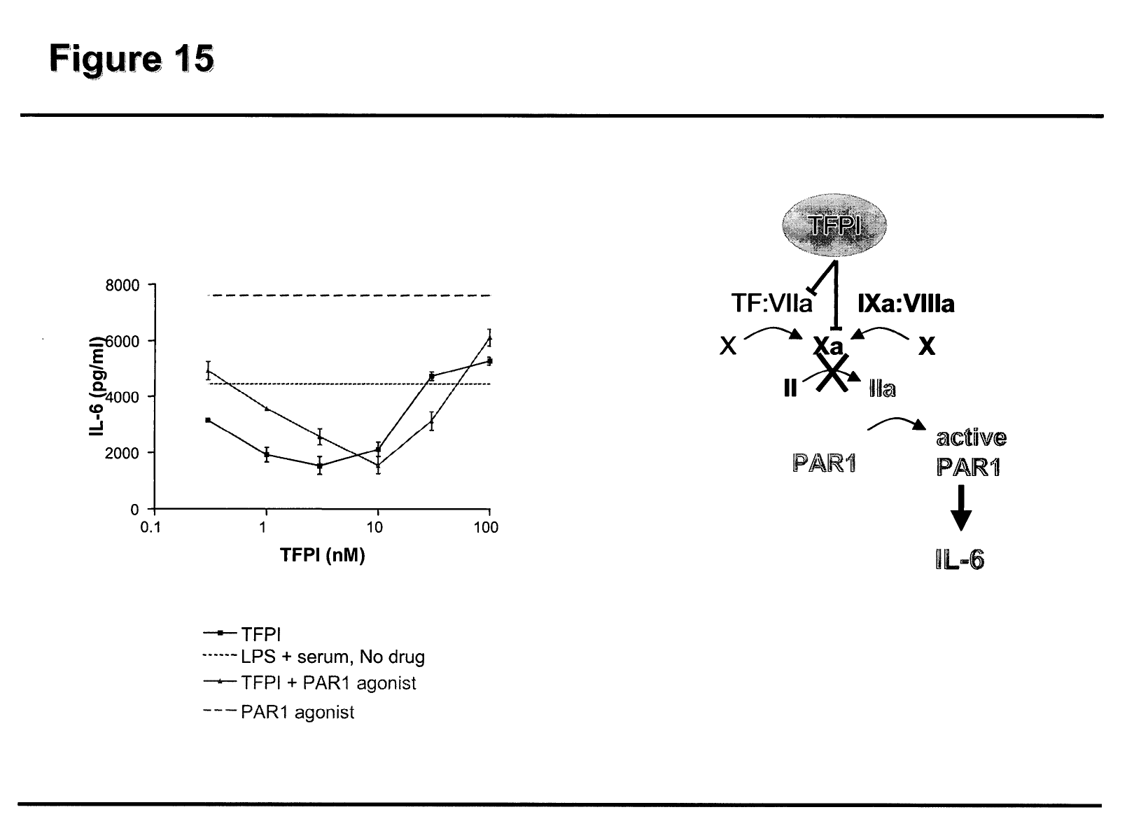

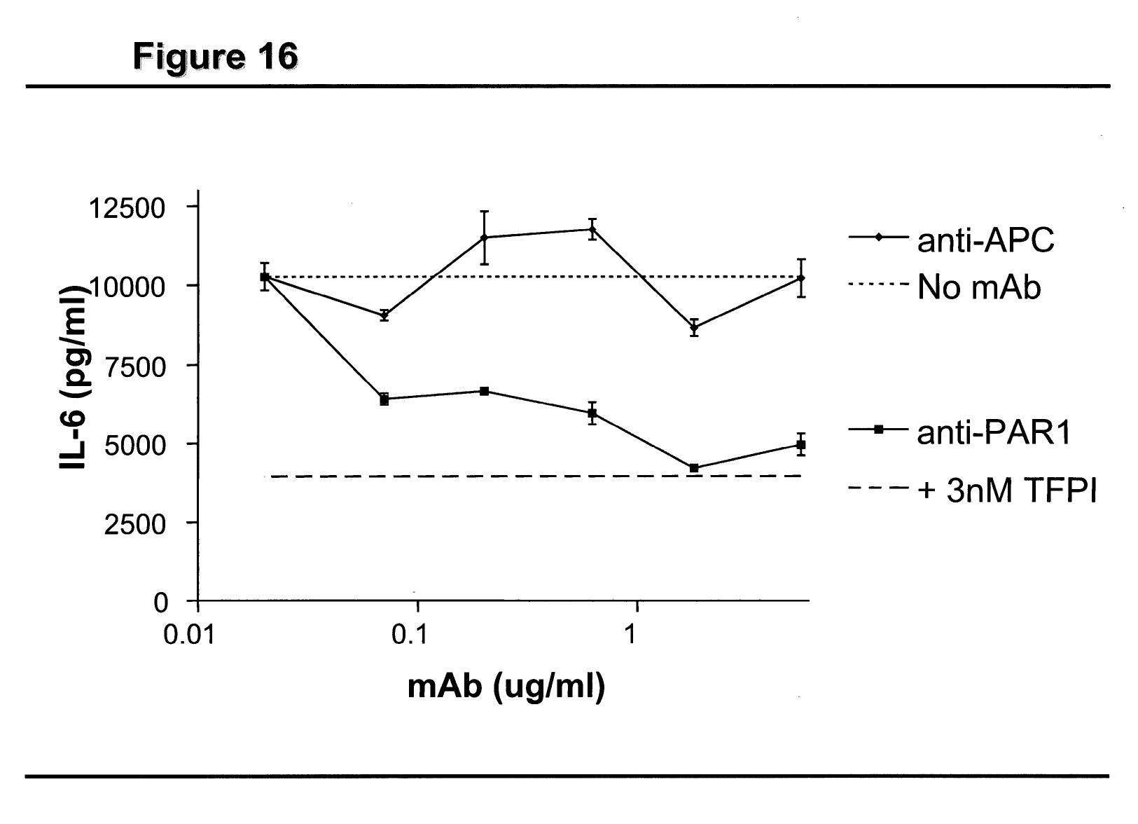

In agreement with this model we found that regulation of protease-activated receptor (PAR)-induced cytokines depends on factor Xa production even when PAR-1 is activated by an agonist peptide (FIG. 15). PAR-1 seems to be part of the signaling system inducing IL-6 secretion in our septic blood model, because an antibody that blocks PAR-1 activation inhibited IL-6 secretion (FIG. 16). Further addition of PAR-1 agonist increased IL-6 secretion. Both of these activities are in agreement with literature data. on PAR being responsible for inflammatory cytokine production. Since PAR activation is downstream of the coagulation cascade, PAR agonists should be dominant in this system. Instead we find that TFPI is able to override PAR agonist in alignment with our model of a Xa dependent TFPI complex regulating cytokines.

### EXAMPLE 13

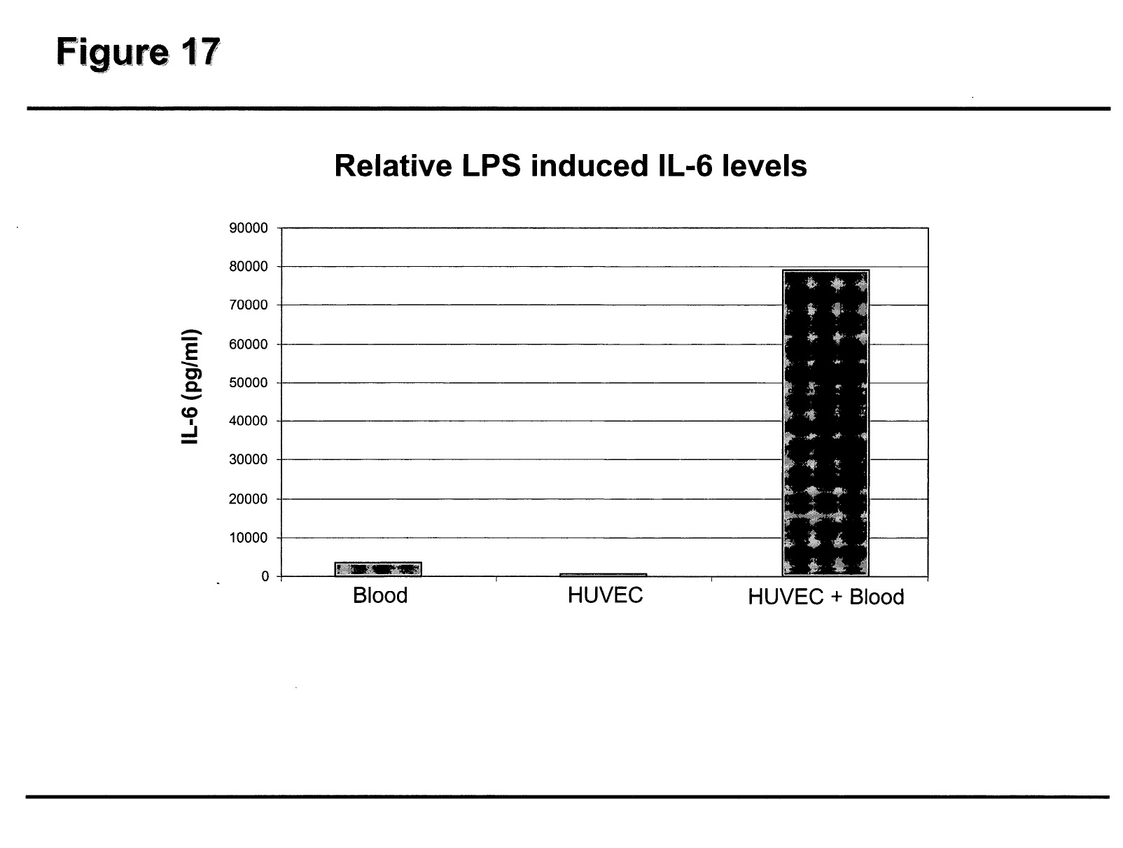

The major reservoir of TFPI in caveolae is found on endothelial cells. These cells are proposed to serve as sentinels for blood infection through their rapid induction of surface TF. As mentioned above, we found that blood responds to LPS by releasing a modest burst of IL-6 (FIG. 17). Similarly, endothelial cells respond to LPS with a small amount of cytokines. We have found that combining blood and endothelial cells leads to a synergistic response to LPS. We determined that the synergistic response was restricted to IL-6 and IL-8; this matches the role of endothelial cells as an alarm system.

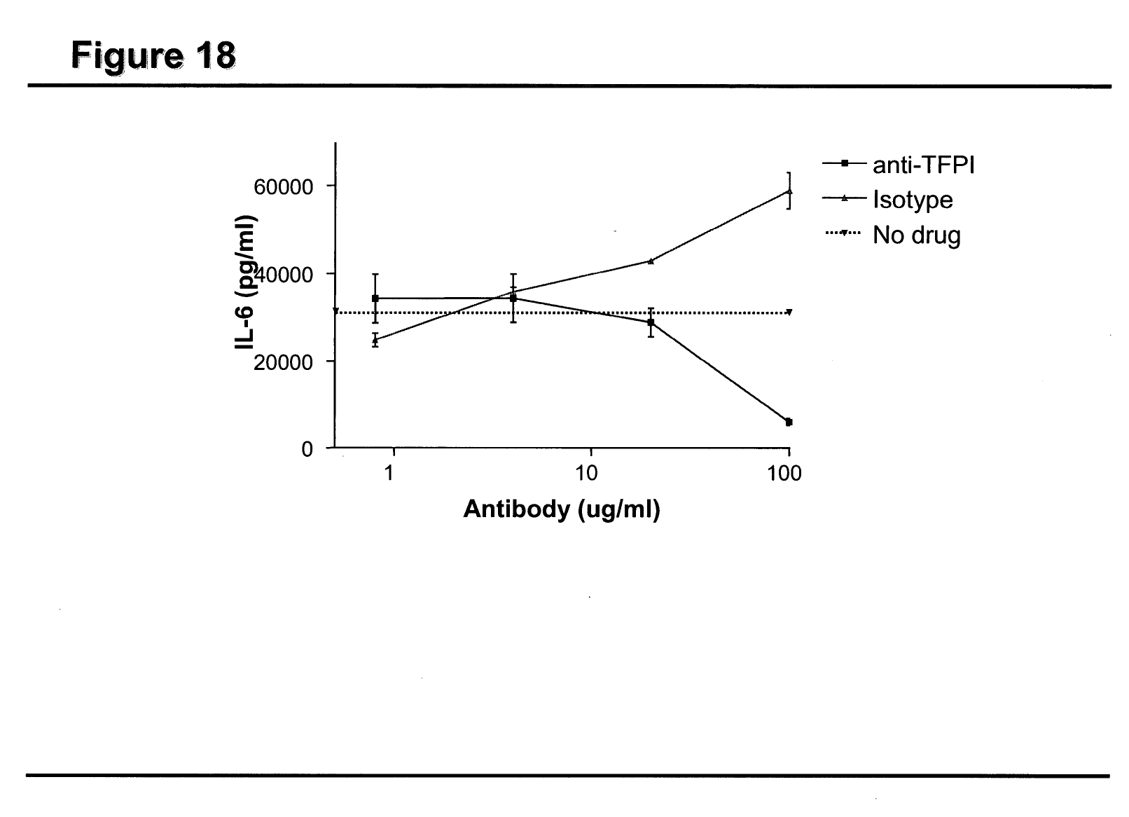

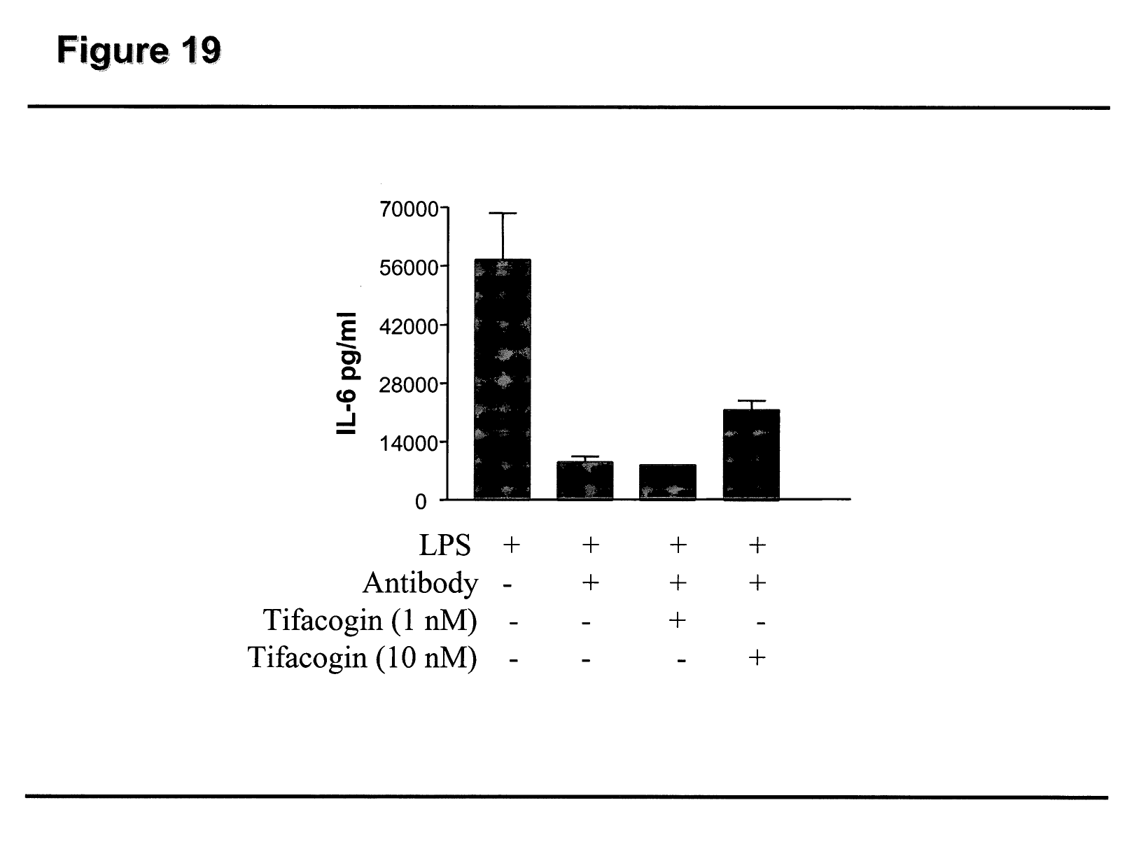

Once again to probe the role of TFPI in this reaction we added neutralizing anti-TFPI antibody (FIG. 18). We found that a high concentration of anti-TFPI inhibits the production of IL-6. To confirm that the inhibition was due to TFPI, we preincubated the anti-TFPI with small concentrations of ala-TFPI and found that 10 nM ala-TFPI partially reversed the effect of 600 nM anti-TFPI (FIG. 19).

1. A method of (1) treating a patient at risk of developing or diagnosed as having a severe bacterial infection or (2) reducing the risk of mortality from a severe bacterial infection, comprising administering TFPI or a TFPI analog to a patient in need thereof who meets one or more of the following criteria:
(a) a blood DL-6 level below 3,200 pg/ml;
(b) an International Normalized Ratio (ESfR) below 2.5;
(c) an acute physiology score (APS) less than 26;
(d) an Acute Physiology And Chronic Health Evaluation (APACHE II) score less than 38; and
(e) a MODS score greater than 18.

2. (canceled)

3. The method of claim 1 wherein the severe bacterial infection causes pneumonia, bacteremia, deep tissue infection, skin infection, soft tissue infection, periodontal infection, peritonitis, surgical infection, or meningitis.

4. The method of claim 3 wherein the severe bacterial infection causes pneumonia and the pneumonia is community-acquired pneumonia, or hospital acquired pneumonia.

5. The method of claim 4 wherein the pneumonia is caused by S. pneumoniae.

6. The method of claim 1 wherein the TFPI or TFPI analog is non-glycosylated.

7. The method of claim 1 wherein less than about 12% of the TFPI or TFPI analog molecules are modified species, wherein the modified species include one or more of the following:
i. an oxidized TFPI or TFPI analog molecule, as detected by reverse phase chromatography;
ii. a carbamylated TFPI or TFPI analog molecule, as detected by cation exchange chromatography;
iii. a deamidated TFPI or TFPI analog molecule, as detected by a Promega ISOQUANT® kit;
iv. a TFPI or TFPI analog molecule that comprises a cysteine adduct, as determined by amino acid analysis;
v. aggregated TFPI or TFPI analog molecules, as detected by size exclusion chromatography; and
vi. a misfolded TFPI or TFPI analog molecule, as detected by non-denaturing SDS-polyacrylamide gel electrophoresis.

8. The method of claim 7 wherein:
(a) less than about 9% of the TFPI or TFPI analog molecules are oxidized;
(b) wherein less than about 3% of the TFPI or TFPI analog molecules are carbamylated
(c) less than about 9% of the TFPI or TFPI analog molecules are deamidated;
(d) less than about 2% of the TFPI or TFPI analog molecules comprise a cysteine adduct;
(e) less than about 3% of the TFPI or TFPI analog molecules are aggregated;
(f) less than about 3% of the TFPI or TFPI analog molecules are misfolded.

9-14. (canceled)

15. The method of claim 1 wherein the TFPI or TFPI analog is administered as a formulation comprising arginine or citrate.

16. (canceled)

17. The method of claim 1 wherein the pharmaceutical composition:
(a) comprises 0.01 to 1.0 mg/ml, 0.01 to 0.8 mg/ml, 0.01 to 0.5 mg/ml, 0.01 to 0.3 mg/ml, 0.01 to 0.2 mg/ml, or 0.01 to 0.1 mg/ml TFPI or TFPI analog;
(b) comprises 150-450 mM, 150-400 mM, 150-350 mM, or 150-300 mM L-arginine;
(c) comprises 0.1-50 mM, 0.1-40 mM, 0.1-30 mM, 0.1-25 mM, 0.1-15 mM, 0.1-10 mM, or 0.1-5 mM L-methionine;
(d) comprises 5-50 mM, 5-45 mM, 5-40 mM, 5-35 mM, 5-30 mM, 5-25 mM, or 5-20 mM sodium citrate buffer;
(e) has a pH of 5.0-6, 5.0-5.8, 5.0-5.7, 5.0-5.6, or 5.0-5.5;
(f) comprises 0.15+−15% mg/ml TFPI or TFPI analog, 300+−15% mM L-arginine, 5+−15% mM L-methionine, and 20+−15% mM sodium citrate buffer at pH 5.5+−15%;
(g) comprises 0.15+−10% mg/ml TFPI or TFPI analog 300+−10% mM L-arginine, 5+−10% mM L-methionine, and 20+−10% mM sodium citrate buffer at pH 5.5+10%;
(h) comprises 0.15+−5% mg/ml TFPI or TFPI analog, 300+−5% mM L-arginine, 5+−5% mM L-methionine, and 20+−5% mM sodium citrate buffer at pH 5.5+−5%;
(i) comprises 0.45+−15% m/ml TFPI or TFPI analog, 300+−15% mM L-arginine, 5+−15% mM L-methionine, and 20+−15% mM sodium citrate buffer at pH 5.5+−15%;
(i) comprises 0.45+−10% mg/ml TFPI or TFPI analog, 300+−10% mM L-arginine, 5+−10% mM L-methionine, and 20+−10% mM sodium citrate buffer at pH 5.5+−10%; and/or
(j) comprises 0.45+−5% mg/ml TFPI or TFPI analog, 300+−5% mM L-arginine, 5+−5% mM L-methionine, and 20+−5% mM sodium citrate buffer at pH 5.5+−5%.

18-27. (canceled)

28. The method of claim 1 wherein the TFPI or TFPI analog is administered by continuous intravenous infusion at a dose rate equivalent to:
(a) administration of reference ala-TFPI at a dose rate of less than about 0.66 mg/kg/hr;
(b) administration of reference ala-TFPI at a dose rate from about 0.00025 to about 0.1 mg/kg/hr and wherein the TFPI or TFPI analog is administered for at least about 72 hours;
(c) administration of reference ala-TFPI at a dose rate from about 0.010 to about 0.1 mg/kg/hr;
(d) administration of reference ala-TFPI at a dose rate between about 0.02 to 0.1 mg/kg/hr;
(e) administration of reference ala-TFPI at a total dose from about 0.024 to about 4.8 mg/kg;
(f) administration of reference ala-TFPI at a dose rate between about 0.02 to about 1 mg/kg/hr; or
(g) administration of reference ala-TFPI at a daily dose from about 0.006 mg/kg to about 1.2 mg/k.

29-31. (canceled)

32. The method of claim 1 wherein the TFPI or the TFPI analog is administered for:
(a) at least about 96 hours;
(b) a period of 10-200 hours;
(c) a period of 10-150 hours; or
(d) a period of 24-96 hours.

33-36. (canceled)

37. The method of claim 1 wherein the patient has not received heparin treatment for at least 8 hours before administration of the TFPI or TFPI analog.

38. The method of claim 1 further comprising treating the patient with activated protein C.

39. The method of claim 1 wherein the TFPI analog is administered and the TFPI analog is ala-TFPI.
